(19) United States
(12) Patent Application Publication
(10) Pub. No.: US 2022/0160938 A1
(43) Pub. Date: May 26, 2022

(54) CROSS-LINKED HYALURONIC ACID HYDROGELS COMPRISING PROTEINS

(71) Applicant: LACERTA TECHNOLOGIES INC., Raleigh, NC (US)

(72) Inventors: Zsombor LACZA, Csopak (HU); István HORNYÁK, Miskolc (HU); Adél HINSENKAMP, Budapest (HU)

(21) Appl. No.: 17/622,732

(22) PCT Filed: Jun. 26, 2020
(86) PCT No.: PCT/US2020/039949

§ 371 Date: Dec. 24, 2021

(30) Foreign Application Priority Data
Jun. 26, 2019 (HU) P1900233

(51) Int. Cl. A61L 27/52; A61L 27/36; A61L 27/20; A61K 47/36; C08B 37/08; C08L 5/08

(52) U.S. Cl. CPC A61L 27/52; A61L 27/3616; A61L 27/20; A61K 47/36; A61L 2430/34; C08L 5/08; A61L 2400/06; A61L 2430/10; A61L 2430/30; C08B 37/0072

(57) ABSTRACT

The invention relates to the field of derivatized cross-linked hyaluronic acid hydrogels having blood-derived proteins linked into their structure, as well as preparation and uses thereof.

### FIELD OF THE INVENTION

The invention relates to the field of derivatized cross-linked hyaluronic acid hydrogels having blood-derived proteins linked into their structure, as well as preparation and uses thereof.

### BACKGROUND ART

Hyaluronic acid (HA) is a linear, non-sulphated glycosaminoglycan consisting of repeating units of D-glucuronic acid and N-acetyl-D glucosamine (Fallacara, Baldini et al. 2018). It exists in a high variety of molecular weight, which influences its physical properties, especially viscosity. Under 10³ kDa (1 MDa, i.e. 10⁶ Da) it is considered to be low molecular weight HA and above 10³ kDa it is called high molecular weight HA (Zhao, Wang et al. 2015). Hyaluronic acid can be found naturally in many parts of the mammalian body (Gokila, Gomathi et al. 2018, Suner, Demirci et al. 2019) in the extracellular matrix, umbilical cord, loose connective tissues (Sall and Férard 2007), synovial fluid, cartilage, tissues of the vitreous humor and the skin (Neuman, Nanau et al. 2015), lung, muscle tissues and brain (Zhao, Wang et al. 2015). It is a water soluble, non-immunogenic, biocompatible and biodegradable material (Gokila, Gomathi et al. 2018) and has many biological functions, among others it can interact with cells via membrane receptor CD44 (Suner, Demirci et al. 2019), and can induce cell aggregation, proliferation, migration and angiogenesis (Turley, Noble et al. 2002, Jooybar, Abdekhodaie et al. 2019). Hyaluronic acid also regulates the process of inflammation promoted regeneration of the tissue (Suner, Demirci et al. 2019). High molecular weight HA was reported to have anti-angiogenic and anti-inflammatory effects, while low molecular weight HA fragments act adversely (Schanté, Zuber et al. 2011).

In December 2003, the first hyaluronic acid (HA)-based dermal filler was approved by the FDA. This was rapidly followed by the development of many other HA-based dermal fillers (Guillen, Karim (2011) WO2011119468(A1)).

Hyaluronic acid degrades rapidly in vivo enzymatically by hyaluronidase. To extend its presence in the body and to improve its mechanical properties, HA can be modified by covalently cross-linking the polymer chains forming a hydrogel (Schante, Zuber et al. 2011). A three-dimensional network can be obtained, which is water insoluble and less sensitive to enzymatic degradation (La Gatta, Schiraldi et al. 2011). There are several chemical cross-linkers, which modify the HA chain by different chemical reactions, for instance glutaraldehyde, divinyl sulfone (DVS) (Lai 2014) and butanediol-diglycidyl ether (BDDE) (Schanté, Zuber et al. 2011) acting on the hydroxyl group, while carbodiimides modify the carboxyl group. Several other strategies have also been used, like heterobifunctional cross-linkers such as 1-ethyl-3-(3-dimethylaminopropyl) carbodiimide hydrochloride (EDC), homobifunctional cross-linkers such as PEGDA; other examples are dithiobis (propanoic dihydrazide) (DTPH), and L-aspartamide (PHEA-EDA), (Slaughter, Khurshid et al. 2009).

It was observed that with increasing cross-linker concentration the degree of cross-linking can be enhanced (Schanté, Zuber et al. 2011), which increases the time of degradation and improves mechanical stability. The cross-linking density determinates the swelling ratio as well, a more cross-linked, strong hydrogel will swell less than a weakly cross-linked hydrogel (Kenne, Gohil et al. 2013). However, an extremely high cross-linker concentration has its drawbacks as well, because chemical cross-linkers are generally toxic and can cause unwanted reactions in larger amounts (Hennink and van Nostrum 2002).

Cross-linked hyaluronic acid hydrogels can be used as scaffolds for soft tissue engineering (Van Tomme, Storm et al. 2008, Hardy, Lin et al. 2015) in cases of soft-tissue defects like congenital malformation, extirpation or trauma (Okabe, Yamada et al. 2009) as HA is a biocompatible and biodegradable material and has stimulatory effects on cell proliferation, migration, extracellular matrix secretion and differentiation (Jooybar, Abdekhodaie et al. 2019).

The scaffolds can serve as a synthetic extracellular matrix with their high water content and soft structure (Slaughter, Khurshid et al. 2009) organizing cells into a three-dimensional architecture (Drury and Mooney 2003). As these scaffolds mimic natural tissues, cells adhere into the three-dimensional network, especially when there are incorporated peptide domains in the hydrogel (Slaughter, Khurshid et al. 2009). Preferably, these scaffolds are remodelled and vascularized by adhering cells (Slaughter, Khurshid et al. 2009, Jooybar, Abdekhodaie et al. 2019).

HA hydrogels can also be used to facilitate wound healing. Normally, the process consists of hemostasis, inflammation, proliferation and remodelling (Deutsch, Edwards et al. 2017, Sahana and Rekha 2018), but in some cases natural wound healing process is hindered or cannot take place and the wound becomes chronic, like diabetic ulcers and pressure ulcers (Shimizu, Ishida et al. 2014, Deutsch, Edwards et al. 2017), which cannot be recovered without external help. In other cases, like severe burns, large skin damage occurs and therefore an appropriate wound dressing is needed. An ideal wound dressing prevents contamination of the wound and maintains adequate moisture but removes excessive exudates (Deutsch, Edwards et al. 2017).

Hyaluronic acid hydrogels could be excellent wound dressings as they create an advantageous environment for wound healing because of their rheological, hygroscopic and viscoelastic properties (Shimizu, Ishida et al. 2014). Besides, high molecular weight HA was reported to have cytoprotective effect and to facilitate cell migration and wound healing (Neuman, Nanau et al. 2015). In animal models HA helped re-epithelialization and led to the formation of new soft tissue in case of full-thickness surgical wounds (Shimizu, Ishida et al. 2014, Neuman, Nanau et al. 2015). Although low molecular weight HA was not reported to have these protective effects (Wu, Chou et al. 2013), it was found to induce angiogenesis following its degradation (Shimizu, Ishida et al. 2014).

Cross-linked high molecular weight hyaluronic acid gels alone were found to be bioinert (Ibrahim, Kang et al. 2010) and cell attachment on and into these gels is low (Ramamurthi and Vesely 2002, Zhang, He et al. 2011). However, cell adherence can be promoted by fabricating hybrid HA scaffolds with gelatine (Zhang, He et al. 2011) or collagen (Hardy, Lin et al. 2015) which are possible strategies to improve performance of the hydrogel or to increase cell adhesion.

Satin et al. [Satin, Alexander, M, Norelli, Jolanta B., Sgaglione, Nicholas A. and Grandel, Daniel A. Grande Cartilage 1-10, 2019] report on the additive effect of combined PRP-HA to reduce the expression of enzymes that contribute to the pro-inflammatory, catabolic phenotype observed in OA; their preparation contributes to chondrocyte proliferation and is thus may be useful to treat OA.

A composite HA hydrogel implant is disclosed in WO09048930A2 wherein three compositions are formed consecutively; the middle one being cross-linked collagen wherein the first and the third one being made of cross-linked HA. In one embodiment the cross-linker is BDDE and in another it is divinyl-sulfone (DVS) wherein the DVS-concentration for forming the first composition is preferably 500-10,000 ppm, more preferably 5000 ppm and the temperature for cross-linking is 50-60° C., wherein the concentration of the HA for forming the first composition may be about 30 mg/ml or higher and the pH between 9-12; and wherein the concentration of the HA solution for the third composition may be 3-15% weight/volume, and preferably 5-10% weight/volume and the pH of 7.0-7.6.

In WO2011014432(A1) a hydrogel formed by the reaction of a hyaluronic acid having from 1-10% of its hydroxyl groups derivatized by reaction with divinyl sulfone with a thiol cross-linker having from two to eight thiol groups is disclosed (Gravett, David M, (2011) WO2011014432(A1)), wherein preferably the hyaluronic acid has a degree of conversion of hydroxyl groups to 2-(vinylsulfonyl)ethoxy groups of about 4-5% per disaccharide repeat unit.

WO2019/155391A1 provides a method of synthesizing cross-linked hyaluronic acids and compositions thereof alone or in combination with PRP/BMC as well as uses thereof in cell culture, skincare and joint preservation. The invention provides in particular a method for the production of a crosslinked gel from at least one first polymer (preferably hyaluronic acid) and performing a second cross-linking step with preferably a second polymer. The crosslinked hyaluronic acid can be combined with platelet concentrate, platelet rich plasma (PRP), a bone marrow concentrate and/or a biomaterial.

WO2019/123259A1 discloses hydrogels based on blood plasma components. The solution relates to bioactive hydrogels, wherein a biocompatible polymer (e.g., hyaluronic acid) can be cross-linked with a plasma-derived element, e.g. human platelet rich plasma, human platelet lysate, human plasma protein, or combinations thereof. This is a one step procedure which is not realistic for certain applications and mechanical properties of the gel are questionable.

Further options include cross-linking chitosan (Miranda, Malmonge et al. 2016), collagen (Hardy, Lin et al. 2015) or silk-fibroin (Gokila, Gomathi et al. 2018), to mention a few strategies. Peptide incorporation into the hydrogel is another way to enhance cell attachment, migration, proliferation, growth and organization (Slaughter, Khurshid et al. 2009). Besides, HA hydrogels can be coated with collagen, extracellular matrix gel, laminin and fibronectin to enhance cellular adhesion (Ramamurthi and Vesely 2002). Protein cross-linking into the gels can be another option to advance cell attachment.

### BRIEF DESCRIPTION OF THE INVENTION

In an aspect, the invention relates to a method for the preparation of a cross-linked hyaluronic acid hydrogel and having blood-derived proteins cross-linked into the structure of said hydrogel, said method comprising
providing a hyaluronic acid also known as hyaluronate (HA) solution (preferably between 5 and 15 w/w %,
contacting said HA solution with a first cross-linker to provide a cross-linking reaction mixture,
cross-linking the HA by a first cross-linker to form a cross-linked HA hydrogel in a first cross-linking step,
optionally carrying out a first processing step for processing the cross-linked gel hydrogel, e.g, sterilization using dry or wet heat, EtO or gamma irradiation
preferably freeze-drying the cross-linked HA hydrogel,
contacting a blood-derived protein composition with the cross-linked HA hydrogel,
cross-linking the blood-derived protein by a second cross-linker into the hydrogel to form a protein-cross-linked hydrogel in a second cross-linking step,
optionally carrying out a second processing step for processing the protein-cross-linked hydrogel, e.g, sterilization using dry or wet heat, EtO or gamma irradiation.

The invention also relates to a method or to the previous method for the preparation of a cross-linked hyaluronic acid hydrogel and having blood-derived proteins cross-linked into the structure of said hydrogel, said method comprising
cross-linking HA by a first cross-linker to form a cross-linked HA hydrogel in a pre-determined three dimensional size, e.g. film, block or spherical shape (first cross-linking step)
optionally processing the cross-linked gel hydrogel, preferably washing and/or equilibrating and sterilization
preferably freeze-drying the cross-linked HA hydrogel,
cross-linking a blood-derived protein into the hydrogel to form a protein-cross-linked hydrogel,
optionally carrying out a second processing step for processing the protein-cross-linked hydrogel, preferably selected from washing, shaping including milling, cutting, homogenization and freeze-drying the hydrogel.

Preferably the process is characterized by the two cross-linking step defined above or herein.

In a preferred embodiment freeze-drying the cross-linked HA hydrogel is important and advantageous as the blood-derived protein is distributed in a high concentration due to the dry sponge-like structure of the freeze-dried matrix. Preferably the process is characterized by the freeze drying step after the first cross-linking step.

Preferably the cross-linking reaction mixture, preferably the first cross-linking reaction (more preferably with 1,4 butanediol diglycidyl ether (BDDE) or divinyl sulfone (DVS), preferably DVS) is provided in alkaline condition; preferably an alkali-hydroxide is added, preferably NaOH. Preferred pH values are defined below.

Preferably pelleting or sedimentation, preferably centrifugation is carried out in or before the first cross-linking step to obtain flat gels which are allowed to cross-link. Preferably centrifugation is carried out at 1000 to 5000 g, preferably at 1000 to 3000 g, more preferably at 1000 to 2500 g, highly preferably at 1500 to 2000 g.

Preferably centrifugation is carried out for 1 to 10 minutes, preferably for 1 to 5 minutes, preferably for 2 to 7 minutes, preferably for 2 to 4 minutes, in particular for about 3 minutes.

In a preferred embodiment centrifugation is carried out at 1000 to 3000 g for 1 to 10 minutes or for 1 to 5 minutes; in a further preferred embodiment centrifugation is carried out at 1000 to 2500 g or at 1500 to 2000 g for 1 to 5 minutes.

In a preferred embodiment the reaction mixture is stirred, preferably vortexed before pelleting and cross-linking.

In a preferred embodiment the hyaluronate is a 0.5 to 2,5 MDa, preferably 1 to 2 MDa, more preferably 1 to 1.4 MDa or 1.2 to 1.4 MDa, highly preferably 1.3 to 1.4 MDa hyaluronate (HA). In a preferred embodiment the hyaluronate is a middle or high molecular weight HA or preferably a middle molecular weight HA.

In a highly preferred embodiment the HA as a starting material is freeze-dried hyaluronate.

In a preferred embodiment the cross-linker is used in 1 to 8 VV %, preferably in 1 to 6 V/V % or in 1 to 5 V/V % or in 2 to 6 V/V %, more preferably in 2 to 5 V/V %. In a highly preferred embodiment the concentration of the cross-linker is 4 to 6 V/V %, preferably about 5 V/V %. Unless indicated otherwise the concentration percentage of the cross-linker is given in V/V %. Highly preferably the cross-linker is DVS.

Preferably the gels are washed after cross-linking by aqueous medium, preferably by water.

Highly preferably the washed gels are sterilized, preferably heated to above 100° C., preferably to 110 to 130° C., preferably autoclaved for 10 to 30 minutes, preferably to 15 to 25 minutes, more preferably to about 20 minutes.

In a preferred embodiment cross-linking is performed at 20 to 29° C., preferably at 20 to 25° C., preferably at about room temperature and ambient pressure.

In a preferred embodiment sterilized gels are freeze-dried. Freeze-drying is preferably carried out from −100 to −20° C., preferably from about −70 to −40° C. e.g. at −51 to −55° C. or at −50 to −60° C., at 0.2 to 20 Pa, preferably at 0.5 to 10 Pa, highly preferably at about 1 to 8 Pa, preferably at 1 to 5 Pa. Freeze-drying is preferably carried out at −55° C. and 5 Pa. Preferably freeze-drying is carried out from about −70 to −40° C., preferably at 0.5 to 10 Pa.

In a preferred embodiment the HA gel so obtained preferably the sterile, freeze-dried HA gel is further modified by cross-linking.

In a preferred embodiment the blood-derived protein is selected from the group consisting of plasma, plasma-preparation, serum, serum-preparation, isolated plasma proteins or isolated plasma protein compositions, prepared by pheresis, centrifugation or cryoprecipitation.

In a preferred embodiment the blood-derived protein is selected from the group consisting of:

a) plasma preparation is selected from activated plasma, pooled plasma and antibody-reduced plasma,

b) the serum preparation is selected from platelet-rich plasma and serum fraction of PRF (SPRF or hyperacute serum) and coagulated whole blood.

c) the isolated plasma protein composition is selected from serum-albumin, serum albumin plus regulatory proteins, serum albumin plus fibrinogen and blood-clotting factors, regulatory proteins plus fibrinogen and blood-clotting factors, serum, plasma, cryoprecipitate; optionally wherein at least a part of the plasma proteins is/are recombinant protein(s).

In a preferred embodiment the blood-derived protein is selected from a serum fraction of platelet-rich fibrin (SPRF) and a fibrinogen preparation.

In a preferred embodiment in a second cross-linking step SPRF is cross-linked using DVS. Preferably, when preparing SPRF containing gels, freeze-dried gel is contacted with 1-6%, preferably 4 to 6%, e.g. about 5% sterile DVS containing SPRF at alkaline pH as taught herein, and crosslinking reaction is carried out as taught herein. The gels were washed to remove excess non-reacted DVS.

In a further preferred embodiment in a second cross-linking step fibrinogen is polimerized. In a preferred embodiment a serum cryoprecipitate is used as fibrinogen. Preferably 0.5 to 2 M Ca²⁺, more preferably 0.5 to 2 M CaCl₂, as well as thrombin is added to said fibrinogen preparation, preferably serum cryoprecipitate, preferably recalcined cryoprecipitate.

Preferably fibrinogen converts into fibrin polymers inside the structure of the HA gels in one hour at room temperature.

Once aseptic conditions are applied there is no need to further sterilize the prepared SPRF and fibrin containing gels.

In an embodiment the HA is a hyaluronate salt, e.g. sodium hyaluronate.

Preferably, said first cross-linker is a cross-linker acting on hydroxyl groups, preferably 1,4 butanediol diglycidyl ether (BDDE) or divinyl sulfone (DVS), preferably DVS.

Preferably, said second cross-linker is a cross-linker acting on hydroxyl groups, preferably 1,4 butanediol diglycidyl ether (BDDE) or divinyl sulfone (DVS), preferably DVS.

Preferably both the first and second cross-linker is DVS.

Preferably the HA solution comprises HA having a molecular weight (MW) of 0.1-10 MDa, preferably middle or high molecular weight (HMW) HA having a molecular weight of 1 MDa or higher, preferably a MW or 1-10 MDa. In a preferred embodiment the hyaluronate is a 0.5 to 2,5 MDa, preferably 1 to 2 MDa, more preferably 1 to 1.4 MDa or 1.2 to 1.4 MDa, highly preferably 1.3 to 1.4 MDa hyaluronate (HA).

Preferably the first cross-linking reaction mixture comprises a cross-linker in 1 to 15% (weight percent or W/V percent). Preferably the cross-linker is BDDE or DVS, particularly preferably DVS, and alkaline pH is provided in the cross-linking reaction mixture. More preferably the alkaline pH is set by an alkaline hydroxide. In a very preferred embodiment the pH is set by NaOH concentration of which in the cross-linking reaction mixture is 0.05 to 1 mol/L. In a preferred embodiment the pH is alkaline pH, e.g. is at least pH 9 and at most pH 14 or 13, preferably the pH is 10 to 13 or pH U to 12 or the pH is U to 13.

Cross-linking is carried out preferably for 12 to 96 hours, preferably for 24 to 72 hours, more preferably for 36 to 56, or to 40 to 56, or to 44 to 52, or to 36 to 52 or to 44 to 56 hours, highly preferably for 48 hours. Cross-linking can be carried out at a broad temperature range, preferably is carried out at room temperature.

The cross-linked hydrogels are preferably thoroughly washed to remove any unreacted compounds. Upon washing it may be buffered or the desired compound is allowed to be transferred into the inner parts.

The cross-linked hydrogels are preferably processed e.g. by forming or placing into a mould or the reaction is carried out in a mould, in particular when a shaped product is desired.

Upon processing the gels, preferably the washed gels are sterilized, preferably autoclaved.

In a highly preferred embodiment the cross-linked hydrogels, preferably the washed and sterilized hydrogels are freeze-dried. Freeze-drying is preferably carried out from −100 to −20° C., preferably from about −70 to −40° C. e.g. at −51 to −55° C., at 0.2 to 20 Pa, preferably at 0.5 to 10 Pa, highly preferably at about 1 to 8 Pa, preferably at 1 to 5 Pa.

In a preferred embodiment the hydrogels are pelleted and allowed to cross-link during the first cross-linking step.

In the second cross-linking step, when blood-derived proteins are cross-linked into the hydrogel, the procedure may be analogous to or identical with the first cross-linking step, optionally mutatis mutandis.

In a preferred embodiment the protein composition is combined with a cross-linker.

In an embodiment the cross-linker is inherently present in the blood-derived protein composition, e.g. preparation, e.g. by a blood-clotting factor or multiple blood-clotting factors. In a preferred embodiment blood clotting factor cross-linkers comprise factor XIII, e.g. factor XIIIa cross-linker. In an embodiment the protein composition is a plasma or a plasma preparation.

In an embodiment Ca-gluconate is used as a cross-linker.

In a group of alternative embodiments the blood-derived protein composition is combined with cross-linkers. Preferred cross-linkers are selected from cross-linkers derivatizing the hydroxy groups or amino groups of the proteins and optionally that of the hydrogels. Preferably the cross-linker is an artificial cross-linker and preferably is BDDE or DVS, particularly preferably DVS.

In a preferred embodiment the freeze-dried gel obtained after the first cross-linking step is further modified by a second cross-linking step.

In a preferred embodiment cross-linking is carried out at an alkaline pH. In a preferred embodiment the pH is at least pH 9 and at most pH 14 or 13, preferably pH 10 to 13 or pH 11 to 12 or pH 11 to 13.

In a preferred embodiment cross-linking takes place for preferably for 6 to 72 hours, preferably for 12 to 48 hours, more preferably for 18 to 36 hours at room temperature.

As a processing stem the gels are preferably thoroughly washed, e.g. with sterile distilled water or equilibrated with water or a buffer to remove contaminants e.g. unreacted cross-linker(s). Upon washing a substance e.g. a medicament can be introduced into the hydrogel. Preferably the protein cross-linking and washing procedure is carried out under sterile conditions.

In a preferred embodiment as a processing step the protein-cross-linked hydrogel is formed or shaped, e.g. by cutting, milling, homogenization, pressing or pressing into moulding. Cutting can be made to form into a special form of the application site or to form sheets. Pressing is typically for preparing membranes or sheets.

The invention also relates to a method wherein the blood-derived protein composition is selected from the group consisting of plasma, plasma-preparation, serum, serum-preparation, isolated plasma proteins or isolated plasma protein compositions prepared by pheresis, centrifugation or cryoprecipitation.

In preferred embodiments the

a) plasma preparation is selected from activated plasma, pooled plasma and antibody-reduced plasma,

b) the serum preparation is selected from coagulated whole blood, platelet-rich plasma and serum fraction of PRF (SPRF or hyperacute serum),

c) the isolated plasma protein composition is selected from serum-albumin, serum albumin plus regulatory proteins, serum albumin plus fibrinogen and blood-clotting factors, regulatory proteins plus fibrinogen and blood-clotting factors, serum, plasma, cryoprecipitate; optionally wherein at least a part of the plasma proteins is/are recombinant protein(s).

In a preferred embodiment the second cross-linker is DVS. In a preferred embodiment the first cross-linker is DVS. In a preferred embodiment both cross-linkers are DVS. In a preferred embodiment the DVS cross-linkers are used in 0.5-15%, preferably in 1-12%, preferably in 1-8%, more preferably in 2%, to 7%, highly preferably in 2% to 5%. In a preferred embodiment the pH in the cross-linking reactions is set to pH 10 to 14, preferably to 11 to 13.

In a preferred embodiment the HA has a MW of from 0.1 to 10 MDa HA, preferably the HA is a middle or high molecular weight HA, highly preferably 1 to 3 MDa HA. Preferably the starting HA is freeze-dried sodium hyaluronate. In a preferred embodiment the hyaluronate is a 0.5 to 2,5 MDa, preferably 1 to 2 MDa, more preferably 1 to 1.4 MDa or 1.2 to 1.4 MDa, highly preferably 1.3 to 1.4 MDa hyaluronate (HA).

In a highly preferred embodiment
in the first processing step the BDDE or DVS-cross-linked gel is sterilized (e.g. autoclaved) and freeze dried, preferably also formed (e.g. poured, moulded, milled or cut into form or pieces);
in the second processing step the protein-cross-linked hydrogel is freeze-dried, and/or milled, and/or sterilized or aseptically produced and/or packaged.

In a further aspect the invention relates to a protein-cross-linked hyaluronic acid hydrogel (protein-cross-linked HA hydrogel) having blood-derived proteins cross-linked into the structure of said hydrogel.

In an embodiment the protein-cross-linked HA hydrogel is obtained or is obtainable by any method disclosed herein for the preparation of such hydrogel.

In an embodiment the starting HA is a hyaluronate salt, e.g. sodium hyaluronate.

Preferably, said first cross-linker is a cross-linker acting on hydroxyl groups, preferably 1,4 butanediol diglycidyl ether (BDDE) or divinyl sulfone (DVS), preferably DVS. Preferably the hydrogel is cross-linked with BDDE and/or divinyl-sulfone (DVS) cross-linker.

Preferably, said second cross-linker is a cross-linker acting on hydroxyl groups, preferably 1,4 butanediol diglycidyl ether (BDDE) or divinyl sulfone (DVS), preferably DVS. Preferably both the first and second cross-linker is DVS. In a preferred embodiment the hydrogel comprises cavities and/or the surface of the hydrogel is rugged or rough.

Preferably the HA chains or polymers in the hydrogel has a molecular weight (MW) of 0.1-10 MDa, preferably high molecular weight (HMW) HA having a molecular weight of 1 MDa or higher, preferably a MW or 1-10 MDa. In a preferred embodiment the hyaluronate is a 0.5 to 2,5 MDa, preferably 1 to 2 MDa, more preferably 1 to 1.4 MDa or 1.2 to 1.4 MDa, highly preferably 1.3 to 1.4 MDa hyaluronate (HA).

The cross-linked hydrogels are formed or shaped or moulded (or the reaction is carried out in a mould). Preferably the hydrogels are in the form of a graft, shaped prostheses, membrane, filler, wound cover etc. preferably the gels are washed and preferably washed gels are sterilized, preferably autoclaved.

Preferably the blood-derived proteins are cross-linked into the hydrogel and thus they interpenetrate the HA hydrogel. In a preferred embodiment the proteins form a protein matrix or mesh or interconnected network. In an embodiment the protein matrix or mesh is also cross-linked to the HA hydrogel. In a preferred embodiment the second cross-linkers are natural cross-linkers inherently present in the blood-derived protein composition, in particular they are selected or they form a mixture of blood clotting factor cross-linkers comprising fibrinogen, fibrin monomer, pro-thrombin, thrombin, and factor XIII, e.g. factor XIIIa cross-linker. In an embodiment the protein composition is a plasma or a plasma preparation.

Further preferred cross-linkers cross-linking proteins are selected from cross-linkers derivatizing the hydroxy groups of the proteins and optionally that of the hydrogels. Preferably the cross-linker an artificial cross-linker and preferably is BDDE or DVS, particularly preferably DVS.

Preferably the blood-derived protein composition is selected from the group consisting of plasma, plasma-preparation, serum, serum-preparation, isolated plasma proteins or isolated plasma protein compositions, prepared by pheresis, centrifugation or cryoprecipitation.

In a preferred embodiment the

a) plasma preparation is selected from activated plasma, pooled plasma and antibody-reduced plasma,

b) the serum preparation is selected from platelet-rich plasma and serum fraction of PRF (SPRF or hyperacute serum) and coagulated whole blood.

c) the isolated plasma protein composition is selected from serum-albumin, serum albumin plus regulatory proteins, serum albumin plus fibrinogen and blood-clotting factors, regulatory proteins plus fibrinogen and blood-clotting factors, serum, plasma, cryoprecipitate; optionally wherein at least a part of the plasma proteins is/are recombinant protein(s).

In a preferred embodiment the blood-derived protein is selected from the group consisting of SPRF and fibrinogen and cryoprecipitated serum or serum product.

In a preferred embodiment said blood-derived proteins are cross-linked into the structure of said hydrogel by DVS and the hydrogel comprises cavities and/or the surface of the hydrogel is rugged or rough.

In an embodiment the protein-cross-linked HA hydrogel the cross-linkers are used in 0.5-15%, preferably in 1-12%, preferably in 1-8%, more preferably in 2%, 5% and 10%, highly preferably in 2% to 5%. Preferably the cross-linker is DVS.

In a preferred embodiment in the protein-cross-linked HA hydrogel the HA has a high molecular weight of 0.1 to 10 MDa, preferably the HA is a high molecular weight HA, highly preferably 1 to 3 MDa.

Preferably the protein-cross-linked HA hydrogel is
freeze dried and/or
   is provided in the form of a powder, film or a freeze-dried formed (optionally moulded) scaffold, preferably in a sponge-like form.

In a preferred embodiment the hydrogel comprises additional medication and used as a medication delivery device. In an embodiment the hydrogel comprises additional scaffold forming material selected from the group consisting of gelatin, chitosan etc. Preferably the hydrogel is a hybrid hydrogel or a composite hydrogel. In an embodiment the hydrogel incorporates peptide to enhance cell attachment, migration, proliferation, growth and organization.

In a further aspect the invention relates to the use of the protein-cross-linked HA hydrogel as obtained according to the invention (in any of the methods of claims) or of the protein-cross-linked HA hydrogel of the invention (as claimed in any of the product claims), for soft tissue implantation, wound healing, internal bleeding or muscle and tendon regenerative material.

In an aspect the invention relates to a method for treatment of a subject with administering, preferably grafting the hyaluronic acid hydrogel (protein-cross-linked HA hydrogel) having blood-derived proteins cross-linked into the structure of said hydrogel. Said hydrogel is any hydrogel as defined above or in the appended claims or any hydrogel obtained or obtainable by a method for the preparation of hydrogels of the invention as defined herein.

In said method the protein-cross-linked HA hydrogel is administered, preferably grafted or implanted into a mammalian, preferably human subject at the site of his/her body to be subjected to regenerative treatment.

In a preferred embodiment the hydrogel is used for soft tissue implantation wherein said hydrogel is implanted into a subject. The hydrogel may be formulated as a homogenized powder and may be applied as a suspension. The hydrogel may be moulded and formulated to have a shape of a graft.

In a preferred embodiment the hydrogel is used in wound healing applications, wherein the hydrogel is provided in the form of a wound cover and applied on the wound. In an embodiment the bleeding is internal bleeding and the hydrogel is applied as an implant or graft at the site of internal bleeding to stop said bleeding.

In an embodiment of the invention natural wound healing process is hindered or blocked. In an embodiment the hydrogel of the invention is used for a wound which is chronic, like diabetic ulcer or pressure ulcers. In a further embodiment the hydrogel is used in wounds which are severe burns wherein large skin damage occurs, as a wound dressing. Preferably, contamination of the wound is prevented. Preferably moisture is maintained by the wound dressing. Preferably excessive exudates are removed by the wound dressing.

In a further embodiment the hydrogel is used in a full-thickness surgical would and helps re-epithelialization and for the formation of new soft tissue.

In a preferred embodiment the hydrogel is used in regenerative medicine. The hydrogel may be formulated as a homogenized powder and may be applied as a suspension. The hydrogel may be moulded and formulated to have a shape of a graft. In a preferred embodiment the hydrogel is used in muscle e.g. in reconstruction surgery or as plastic surgery. In a preferred embodiment the hydrogel is used as tendon regenerative material.

Crosslinked hyaluronic acid hydrogels can be used in the method of the invention as scaffolds for soft tissue engineering. Soft tissue defects may be due to congenital malformation, extirpation or trauma, etc. The hydrogel may be formulated as a homogenized powder and may be applied as a suspension. The hydrogel may be moulded and formulated to have a shape of a graft. For example, the scaffolds can serve as a synthetic extracellular matrix organizing cells into a three-dimensional architecture. As these scaffolds closely mimic natural tissues, cells adhere into the three-dimensional network, especially when there are incorporated peptide domains in the hydrogel.

### Definitions

The term “hyaluronic acid” refers to a polymer comprising repeated disaccharide subunits of hyaluronan consisting of D-glucuronic acid and D-N-acetylglucosamine unit. A hyaluronic acid is meant to encompass naturally occurring hyaluronic acid (also referred to as hyaluronan), derivatized hyaluronic acid, e.g. derivatized at one or more positions of the disaccharide subunit, salt forms, hyaluronic acid linker complexes, and hyaluronic acid conjugates. The term “natural hyaluronic acid” or “hyaluronan” is meant to refer to unmodified or non-derivatized hyaluronic acid. Preferably hyaluronic acid is hyaluronan which may be used, in an embodiment, as a starting material of the invention.

The terms “hyaluronic acid derivative” or “derivatized hyaluronic acid” or “modified hyaluronic acid” refers to hyaluronic acid that has been derivatized by reaction with, e.g., one or more small chemical moieties such as divinyl sulfone or the like.

A “hydrogel” as used herein is a colloid network of polymer chains that are hydrophilic and contains or is capable of containing water; preferably the network is formed from the polymer chains by cross-linking.

A “hyaluronic acid hydrogel” or “cross-linked hyaluronic acid hydrogel” refers to a hydrogel which comprises hyaluronic acid polymer chains cross-linked by a small chemical cross-linker.

A sulfonyl-cross-linked hyaluronic acid hydrogel refers to a hyaluronic acid polymer as described above having three or more disaccharide repeat units and comprising at least one sulfonyl bond.

The epoxy-cross-linked hyaluronic-acid hydrogel contains hyaluronic chains cross-linked with a cross-linker comprising at least one epoxy bond.

“Blood-derived protein” as used herein is understood as a composition comprising one or more proteins which is/are derived from blood or plasma (“plasma-derived protein”) or serum (“serum-derived protein”).

“Blood plasma” is a component of blood that does not comprise the blood cells and which is prepared by removing, preferably by centrifugation, the cellular elements of blood, however, which comprises blood clotting factors and which is capable of clotting. The “blood plasma”, as a blood component, is the liquid part of the blood that carries cells and proteins, provides medium for the blood cells in whole blood in suspension and in its isolated form, as preferably understood herein, it is in the form wherein at least the blood cells are removed. Optionally blood plasma comprises an anticoagulant and preferably in the present invention it is reactivated to enable coagulation to form a fibrin matrix.

“Blood serum” is blood plasma without clotting factors or blood plasma made incapable of clotting by removing clotting factors or is a supernatant that is separated using centrifugation after whole blood clotting. As a blood component the serum is neither a blood cell nor a clotting factor, like fibrinogens. Thus, serum does not contain white blood cells (leukocytes), or red blood cells (erythrocytes), Serum comprises, however, proteins not used in blood clotting and all the electrolytes, antibodies, antigens, hormones. Serum is typically prepared by removing blood cells, platelets (or thrombocites) and fibrinogens. Preferably, blood is centrifuged to remove cellular components. (Historically, anti-coagulated blood yields plasma containing fibrinogen and clotting factors. Coagulated blood (clotted blood) yields serum without fibrinogen, although some clotting factors remain.)

“Fetal bovine serum” (FBS) is the serum obtained from the blood drawn from a bovine fetus via a closed system of collection; FBS comprises a very low level of antibodies and contains a high level of growth factors. FBS is a widely used serum-supplement for the in vitro cell culture of eukaryotic cells.

“Human serum albumin”, or alternatively blood albumin, is a globular protein called albumin found dissolved in vertebrate blood and is the most abundant blood protein in mammals. Human and mammalian albumin is encoded by the ALB gene or an ortholog thereof. Serum albumin is produced by the liver.

The term “platelet rich plasma” or PRP is herein understood as a volume of plasma that has a platelet concentration above baseline. Normal platelet counts in blood range between 150,000/microliter and 350,000/microliter. The platelet concentration is specifically increased by centrifugation, and/or otherwise fractionation or separation of the red blood cell fraction, e.g. centrifugation of whole blood first by a soft spin such as 8 min at 460 g and the buffy coat is used or further pelleted by a hard spin at higher g values. PRP typically comprises an increased platelet concentration, which is about a 1.5-20 fold increase as compared to venous blood.

Alternatively, “Platelet rich plasma” (PRP) is a blood fraction prepared by separating the red blood cell fraction from a venous blood sample e.g. a venous blood sample, removing the red blood cell fraction and, if appropriate, the buffy coat, obtaining thereby a platelet poor plasma fraction (PPP), separating—preferably by centrifugation—a platelet rich fraction from the PPP or pelleting platelets, and recovering the platelets in a platelet rich plasma (PRP) fraction, optionally by resuspending the pelleted platelets in an appropriate medium, optionally in PPP.

Such centrifugation and/or fractionation will separate the red blood cells from the other components of blood, and further separate the platelet rich fraction (PRP) including platelets, with or without white blood cells together with a few red blood cells from the platelet poor plasma. PRP may be further concentrated by ultrafiltration, where the protein content of the platelet-rich plasma is concentrated from about 5% to about 20%.

PRP of the prior art typically comprises anticoagulant and clotting is carried out by a clotting agent. However, platelet rich plasma may be prepared by centrifuging blood without an anticoagulant and may be activated upon preparation.

“Platelet rich fibrin” (PRF) is clotting spontaneously during its preparation by centrifuging a blood sample, preferably accelerated upon contact with negatively charged surfaces and with or without adding exogenous coagulation activators.

Preferably, the serum fraction of PRF (SPRF) may be obtained from a blood sample from a single donor or from multiple donors and mixed together to obtain a single blood sample. According to a specific aspect, the SPRF is obtained from venous blood. Preferably, the SPRF is prepared herein without exogenous anticoagulants that are commonly used in the prior art when preparing PRP, thereby an effective activation of platelets and a content of an activated platelet releasate in the isolated serum fraction is obtained according to the invention.

Preparation of SPRF is described e.g. in WO2014126970, WO2017152172, WO2017193134 and patent publication of the respective patent families. Also SPRF or any PRF exudate can be prepared by a device taught in WO2017093838.

The term “administration” as used herein shall include routes of introducing or applying the hydrogel of the invention, such administration can be topical i.e. may be applied to a particular place on or in the body. When the hydrogel of the invention is administered inside the subject's body it can be performed by invasive surgery, preferably by minimally invasive methods. Preferred administration is grafting or implanting.

The term “subject” as used herein refers to vertebrate animal, preferably a warm-blooded mammalian, particularly a human being. In a particular embodiment the hydrogel of the invention is to be administered to a subject. The use of the hydrogel may be e.g. medical or cosmetic.

The term “patient” includes a subject that receives or is considered to receive either therapeutic treatment to restore healthy or improve a disease condition and prophylactic treatment including maintaining health or improving a healthy condition. The term “treatment” is thus meant to include both prophylactic and therapeutic treatment, in particular to treat, repair or augment a tissue at a target site.

“Stem cells” are undifferentiated or partially differentiated cells with a strong potential to differentiate into several or multiple differentiated cell types and which are also capable of a limited number of cell division to maintain themselves. Thus, stem cells have a limited capability to proliferate and a high potential to differentiate.

“Adult stem cells” (“somatic stem cells” or “tissue stem cells”) are partially differentiated stem cells capable of proliferation, self-renewal, production of a large number of differentiated functional progeny, and are capable of regenerating tissue after injury and having a flexibility in the use of these options.

“Mesenchymal stem cells” (MSCs) are stem cells of stromal origin and/or localization which have the potential to differentiate into several cell types, and are
adherent,
capable of differentiation into mesenchymal tissue, preferably bone, cartilage or adipose tissue in vitro, and preferably are
CD105, CD73 and CD90 positive, do not carry surface markers of blood progenitor cells or heamatopoietic stem cells, and preferably are CD45, CD34, CD14, CD11b, CD79a and CD19 negative.

The term “comprise(s)” or “comprising” or “including” are to be construed herein as having a non-exhaustive meaning and to allow the addition or involvement of further features or method steps or components to anything which comprises the listed features or method steps or components. Such terms can be limited to “consisting essentially of” or “comprising substantially” which is to be understood as consisting of mandatory features or method steps or components listed in a list, e.g. in a claim, whereas allowing to contain additionally other features or method steps or components which do not materially affect the essential characteristics of the use, method, composition or other subject matter.

As used in this specification and the appended claims, the singular forms “a”, “an” and “the” include plural references, and should be construed as including the meaning “one or more”, unless the content clearly dictates otherwise. In general, it is also to be understood that the terminology used herein is for the purpose of describing particular embodiments only, and is not intended to be limiting.

### Abbreviations

ACS (autologous conditioned serum)

BDDE (1,4 butanediol diglycidyl ether)

BM-MSCs (bone marrow derived mesenchymal stem cells)

DVS (divinyl sulfone)

FBS (fetal bovine serum)

FGF (fibroblast growth factor)

MSC (mesenchymal stem cell)

OA (osteoarthritis)

PPP (platelet poor plasma)

PRF (platelet rich fibrin)

PRP (platelet rich plasma)

SPRF (serum fraction of platelet rich fibrin)

### BRIEF DESCRIPTION OF THE FIGURES

FIG. 1: The cross-linked hyaluronic acid gels. 2% BDDE (a), 5% BDDE (b), 2% DVS (c) and 5% DVS (d) containing cross-linked hydrogels.

FIG. 2: The swelling ratio. The quotient of the swollen and the freeze-dried gels' weight was higher of BDDE containing cross-linked hydrogels than DVS containing gels. 5% cross-linker containing gels were less swollen than 2% cross-linker containing gels. (n=8)

FIG. 3: The enzymatic degradation of cross-linked hyaluronic acid gels. 2% DVS containing gels degraded the fastest, while 5% cross-linker containing gels were more resistant against enzymatic degradation. Absorbance is proportional to NAG concentration. (n=3)

FIG. 4: The surface (a) and the cross section (b) of the cross-linked HA gels visualized by SEM. The surface of the BDDE cross-linked gels is smooth and their structure is homogenous, while the surface of the DVS gels is rough and they contain bubbles in their structure.

FIG. 5: Cytotoxicity measurement of the cross-linked hydrogels. Cytotoxicity was determined by viability measurement of human MSCs. There is no significant difference between the sample groups and the control group, so, none of the gels is cytotoxic. (n=4)

FIG. 6: MSC attachment onto the DVS cross-linked gels on the 14th day. The cells adhered and proliferated on the blood derived protein containing HA gels. More cells can be seen on SPRF containing gels (b) than on HSA containing gels (a).

FIG. 7: FT-IR spectra of cross-linked hyaluronic acid gels with SPRF (continuous line) and without SPRF (dashed line). Spectra of Na-hyaluronane is also added (dotted lines). On the figure, respectively, spectra of 2% BDDE (a), 5% BDDE (b), 2% DVS (c) and 5% DVS (d) containing cross-linked hydrogels are shown.

FIG. 8: Microscopic image of homogenized gel suspension prepared according to Method 3, as described in Example 1, section 1.1. A: initial gel still containing crystalline type HA parts; B: gel implant obtained from mice after 12 weeks.

FIG. 9: Photos of fibrin containing and control gels (prepared without fibrin) implanted subcutaneously into mice and obtained from mice after 12 weeks. A: fibrin containing gel at the site of implantation. B: control gel at the site of implantation. C. fibrin containing gel isolated from mice. D. control gel isolated from mice.

### DETAILED DESCRIPTION OF THE INVENTION

Hyaluronic acid is an outstanding base material for preparing scaffolds, as it naturally occurs in many parts of the human body, because among others it is water-soluble, biocompatible, biodegradable, resorbable and has regulative roles in angiogenic and inflammatory passages, proliferation and cell motility (Salwowska, Bebenek et al. 2016). To extend its presence in the body when implanted, HA can be chemically modified with different cross-linker reagents. The functional groups available for cross-linking are the hydroxyl group, which may be cross-linked via an ether linkage, and carboxyl groups which are suitable to form an ester linkage.

In most commercial products, HA is cross-linked and the industry standard cross-linking agent is 1,4-butanediol diglycidyl ether (BDDE) in the majority of the market-leading HA fillers as it has been proven to be stable, biodegradable and safe for more than 15 years ahead of other cross-linkers such as divinyl sulfone (DVS) and 2,7,8-diepoxyoctane (De Boulle, Koenraad et al., 2013).

The reaction scheme of DVS cross-linking is shown on scheme 1.

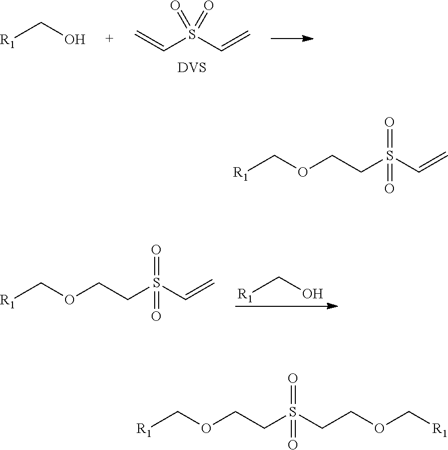

The reaction scheme of BDDE cross-linking is shown on scheme 2.

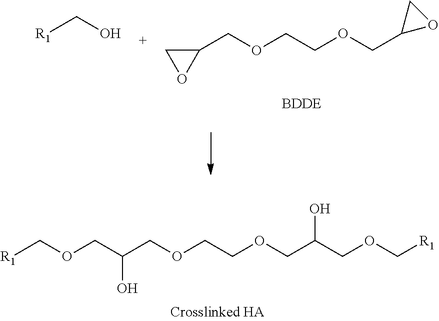

The present inventors have prepared as examples non water-soluble HA hydrogels using BDDE and DVS as cross-linkers in different ratios (2, 5 and 10%) and the effect of different cross-linking agents and cross-linker concentrations were examined on the swelling ratio, resistance against enzymatic degradation and structure of the hydrogels. The non water-soluble HA gels with two different cross-linking reagents, DVS and BDDE, used in 2% and 5% concentration were compared to each other and the strength of the cross-linking was determined by swelling ratio measurement and degradation induced by hyaluronidase.

It has been observed that the cross-linking density increases with the cross-linker concentration (Ghosh, Shu et al. 2005). Besides, it was found, that the water uptake capacity of DVS cross-linked gels is lower than that of the BDDE cross-linked gels, thus for the purpose of the invention DVS is a more effective cross-linker reagent than BDDE. Consequently, the mechanical strength of DVS cross-linked gels may also be greater. The speed of enzymatic degradation is an important property of the gels if the aim is to produce a biodegradable scaffold, which is remodelled by the surrounding cells, however, it is not resorbed until the new tissue is formed. It was found that strongly cross-linked gels, which contain 5% cross-linker degraded slower than 2% DVS or BDDE containing gels. Although, 2% DVS gel was found to be stronger than 2% BDDE gel based on the swelling ratio measurement, 2% DVS containing gel degraded the fastest.

Other methods to prepare DVS cross-linked HA are available in the art like those described in WO2011014432(A1) or by Maiz-Fernández, Sheila et al. (Maiz-Fernández 2019).

The cross-linking reaction with BDDE and DVS are carried out in alkaline pH. For example with DVS the lower limit of the pH is defined by the reaction requirement of a pH higher than 9, and the upper limit was by HA degradation by alkaline hydrolysis (Shimojo, A A M. 2015).

The surface and the cross-section of the cross-linked gels were analyzed by scanning electron microscopy. The gels which were cross-linked with BDDE had a completely smooth surface and homogenous structure, which was not found to promote cell-adhesion onto the gels, while the surface of DVS gels was rougher and they contained many small bubbles inside their structure; this feature was apparently more favorable for cell-adhesion.

Cross-linked HA gels alone do not benefit cell adhesion on the gels and thereby tissue remodelling (Ramamurthi and Vesely 2002, Ibrahim, Kang et al. 2010, Zhang, He et al. 2011), so they are not applicable as scaffolds. In the present invention blood-proteins are linked into the structure of the hydrogel to improve cell attachment onto the gels. In an example, human bone marrow derived mesenchymal stem cells (MSCs) were used to determine the cytotoxicity and biocompatibility of the gels.

As most of the cross-linker reagents are toxic materials, it is important to verify that the cross-linked gels are not cytotoxic. Cytotoxicity was examined culturing human MSCs together with pieces of cross-linked gels and measuring viability. It was concluded that none of the gels was cytotoxic, thus, they could be used for further experiments with MSCs. By performing live-dead staining it was also observed that MSCs are capable of attaching onto the rough surface of DVS gels if they contain SPRF or HSA.

Thus, the present inventors succeeded in preparing non water-soluble cross-linked HA hydrogels, which are in varying degrees resistant against enzymatic degradation, but still biodegradable. Crosslinked HA may degrade slowly, eventually it degrades in a year or longer period. The gels are not cytotoxic and after cross-linking proteins into their structure the ones cross-linked with DVS alone or in combination with BDDE induce cell adhesion, thus proving biocompatibility.

Proteins Linked into the Hydrogels

In the present invention blood derived proteins are linked, particularly cross-linked into the hydrogel of the invention. It is preferred if protein molecules are present in the inner parts of the hydrogel. In a preferred embodiment the cross-linked HA matrix has a structure wherein the density of cross-linked sites allows the migration of proteins into the hydrogel. The second cross-linking step can be performed on the cross-linked hydrogel cross-linked with the first cross-linker in the first cross-linking step.

In the present invention it is advantageous if the proteins can migrate into the hydrogel which can be regulated by cross-linking density; alternatively the majority of the proteins are linked into the structure of the hydrogel. In the preferred embodiment when the proteins diffuse into the inner part or bulk of the hydrogel cross-linked gel produced in the first cross-linking step, the distance, i.e. the space formed between HA polymer strands and cross-linkers shall allow the migration of proteins within the network. Thus, the proteins permeate the hydrogel. Preferably the cross-linked blood-derived proteins form a protein network themselves. This protein mesh, i.e. a network of proteins preferably is integrated into the cross-linked hydrogel. In an embodiment the protein network interpenetrates the cross-linked hydrogel or hydrogel network or mesh.

In a variant the hydrogel network and the protein network are also linked to each other provided by the cross-linker that is capable of binding both functional groups of the protein and of the HA hydrogel, and under the second cross-linking reaction both the proteins and the hydrogel are derivatized.

In a preferred embodiment the hydrogels are freeze-dried after the first cross-linking step and then soaked into a solution of blood-derived proteins or a reaction mixture comprising blood-derived proteins. Thus, the blood-derived proteins permeate the hydrogel network and migrate or diffuse into the inner space of the hydrogel.

In this embodiment when the hydrogel or the HA content of the hydrogel is decomposed in vivo the proteins in the inner part or bulk of the protein-cross-linked hydrogel became available to cells of the subject and the positive cell-recruiting or adhering and proliferating effect of the hydrogel is maintained.

These blood derived proteins can be obtained for example from activated (e.g. recalcified) plasma, pooled plasma, antigen and/or antibody reduced plasma, or from serum, antibody reduced serum, e.g. allogenic (antibody reduced) serum, PRP. In a preferred embodiment the blood derived proteins include factors enabling clotting. The blood-derived proteins of the invention are preferably blood plasma (or shortly plasma) derived proteins. In a highly preferred embodiment the proteins are obtained by cryoprecipitation of plasma.

Preferably, blood-derived proteins of the invention are obtained from blood plasma preparations or blood serum preparations. A blood-derived protein composition is a product comprising blood-derived protein and is suitable for the use in the present invention, i.e. to be linked into the structure of the hydrogel. Wherein it is mentioned that a blood-derived protein is linked into the structure of the hydrogel it is understood to include working with a blood derived protein composition.

In the present examples human serum albumin (HSA) and serum from platelet rich fibrin (SPRF) were cross-linked into the HA hydrogels to induce cell attachment. Surprisingly, DVS was found to be superior for the purposes of the present invention and is used to cross-link HAS and SPRF in preferred embodiments. In an example, human bone marrow derived mesenchymal stem cells (MSCs) were used to determine the cytotoxicity and biocompatibility of the gels.

Serum albumin is an example for a fraction of proteins derived from blood or serum comprising a relatively homogenous composition or a single type of blood-derived protein. Albumin is highly abundant in the blood and functions as a carrier of several substrates. It is also known to have positive effect in regenerative surgery (Skaliczki, Gábor 2013). Thus, protein fractions of blood like albumin are good candidate for blood-derived proteins useful in the present invention.

Nevertheless, the present inventors have found that hydrogels comprising cross-linked SPRF could recruit more cells and cell attachment was stronger. Thus, in terms of cellular effect blood or serum fraction in particular those comprising platelet factors are advantageous. While using SPRF is clearly a preferred option alternatives like platelet-rich plasma or various other PRF exudates (like injectable platelet-rich fibrin), prepared at low g-force e.g. by the Choukroun method can be used (Choukroun J. 2018).

In a further embodiment of the invention natural cross-linking capabilities of blood-derived proteins, in particular fibrinogen is utilized. Plasma contains fibrinogen and clotting factors. Historically, anti-coagulated blood yields plasma whereas coagulated blood (clotted blood) yields serum without fibrinogen, although some clotting factors remain.

In an embodiment blood-derived proteins from plasma are used including fibrinogen. Cross-linking is carried out into the hydrogel by clotting factors. In this embodiment fibrin polymerization from fibrinogen is involved or is the second cross-linking step. Fibrin polymerization comprises several reactions. Fibrin polymerization is initiated by the thrombin cleavage of fibrinopeptides A (FpA) and B (FpB) from the N-termini of the Aα- and Bβ-chains of fibrinogen to produce fibrin monomer (Weisel, J W 2013). Polymerization occurs via protofibrils and later on fibers are formed. Many steps of the highly complex polymerization process are known; however, the precise mechanisms, particular structures, and driving forces supporting the lateral aggregation of protofibrils remain largely unknown. Protofibrils associate with each other laterally to make thicker or thinner fibers only when they reach a threshold length. The fibrin clot or gel exists once the branching fibers form a 3-dimensional network. During and after the polymerization process fibrin is covalently cross-linked by factor XIIIa, also activated by thrombin (Weisel, J W 2013). Thus, factor XIIIa is the major cross-linking agent when fibrinogen as a blood-derived protein is applied in the invention as it catalyzes intermolecular cross-linking of fibrinogen. Already early studies on mixtures of fibrinogen and fibrin indicated factor XIIIa had near equal affinities for the two substrates and the speed and process of polymerization is dependent upon concentration of fibrinogen, fibrin and factor XIII (Kanaide H 1975).

In a preferred embodiment blood-derived protein composition is a blood plasma (plasma) preparation. Plasma is usually prepared by removing, preferably by centrifugation, the cellular elements of blood. As plasma comprises blood clotting factors and is capable of clotting, usually anticoagulant is added for storage. In an embodiment of the invention it is preferred if coagulation occurs to some extent e.g. a fibrin network (i.e. to form a fibrin matrix) is formed in the structure of the hydrogel. In this embodiment plasma, once anticoagulated, is to be re-activated before cross-linked onto the surface of the hydrogel. The hydrogel is preferably a BDDE and/or DVS cross-linked hydrogel, DVS being more preferred.

As an example, blood plasma is separated from the blood by spinning a tube of fresh blood containing an anticoagulant in a centrifuge until the blood cells fall to the bottom of the tube. The blood plasma is then poured or drawn off.

It is to be noted that the use of any blood-derived protein fraction is contemplated in the present invention.

Typically plasma and serum contains dissolved proteins (6-8%) (e.g. serum albumins, globulins, and fibrinogen), glucose, electrolytes (Na+, Ca2+, Mg2+, HCO3—, Cl—, etc.) and hormones etc.

For example blood plasma and/or blood serum comprises the following types or fractions of proteins.

Albumin (more than 50%), present in serum and plasma which has multiple roles in tissue growth and healing, functions as a transporter.

Globulins (35-40%), like alpha-1-globulin fraction and alpha-2-globulin fraction, the beta-globulin fraction and the gamma globulin fraction and are present in the serum and the plasma as well. The gamma-globulin fraction comprises antibodies. In a preferred embodiment the blood-derived protein composition is free of antibodies or gamma-globulins, in particular if it is intended to allogenic use.

Both serum and plasma comprise regulatory proteins like cytokines and growth factors. Typically their ratio is 1% or less in the plasma or serum. However, inclusion of such factors into the blood-derived protein composition is or may be highly advantageous. In an embodiment the ration of pro-inflammatory factors is to be limited e.g. by handling of blood, e.g. by careful, fast and mild handling.

Plasma also comprises fibrinogen, the ratio of which is about as high as 7% in plasma, as well as clotting factors (less than 1%) enabling fibrinogen to be converted into a fibrin network. Formation of a fibrin network or matrix on the surface of the hydrogels of the invention is preferred.

The above serum or serum and plasma components can be used in a separated form as blood-derived proteins or protein compositions of the invention. Alternatively a mixture thereof can be used as well.

For example serum albumin can be used in an isolated form.

In a further embodiment blood-derived protein composition may be blood serum (serum) or a serum derived composition, wherein serum may be considered as blood plasma without clotting factors or blood plasma made incapable of clotting by removing clotting factors. In this case while a fibrin network cannot be formed, however, useful blood-derived proteins are added and facilitate cell recruition upon using the blood-derived protein containing hydrogels of the invention.

In an embodiment the serum derived composition are serum products which also can be used. Such serum products are known in the art.

Fetal bovine serum (FBS) is a widely used serum product and is a supplement for the in vitro cell culture of eukaryotic cells and can be used herein. Also blood-derived protein containing FBS-alternatives may be used.

For example, human platelet lysate (or hPL) is a commercially available substitute supplement for fetal bovine serum (FBS) in experimental and clinical cell culture (see e.g. SIGMA-ALDRICH, PLTMax or STEMCELL Technologies hPL). It is typically obtained from human blood platelets after freeze/thaw cycle(s) that cause the platelets to lyse, releasing a large quantity of growth factors necessary for cell expansion.

As mentioned above, platelet-rich plasma (PRP) can be used which is obtainable from various sources. In a still other preferred embodiment SPRF can be used. Preparation of SPRF is disclosed herein as well as in WO2014126970, WO2017152172, WO2017193134 and patent publication of the respective patent families. Also SPRF or any PRF exudate can be prepared by a device taught in WO2017093838.

The proteins in the protein preparations may also be cell culture proteins. Cell culture proteins are proteins which are or which can be used in cell cultures and as such are non-toxic, compatible with cells and are useful in culturing cells. In a particular embodiment the proteins are different from antibodies. Cells are preferably mammalian cells, more preferably human dells. The proteins are preferably types which support cell growth or cell attachment.

As a further example a combination or a blend of serum-derived proteins can be used. For example a composition comprising albumin and regulatory proteins like a cocktail of cytokines and growth factors can be used. In a still further embodiment, a blood-derived protein like albumin, (or albumin plus selected globulins) plus fibrinogen and clotting factors, optionally completed by regulatory proteins is used.

In an embodiment blood-derived proteins include recombinant variants of such proteins. For example, in humans and mammals albumin is encoded by the ALB gene. Recombinant preparation of blood-derived proteins is well known in the art (J S Powell 2009, M Franchini—2010).

In the present examples human blood-derived proteins, SPRF and HSA were cross-linked with DVS into the structure of the gels to improve cell attachment onto the gels.

Serum from platelet rich fibrin (SPRF, also referred to as hyperacute serum) is a human blood derivative, which is isolated from whole blood without anticoagulants. After blood drawing whole blood is immediately centrifuged in the presence of some glass surface to promote natural blood clotting and gets separated into two fractions, the red blood cell containing fraction and the serum containing fibrin clot. The serum can be squeezed out from the fibrin matrix and that is called SPRF (Kardos, Hornyak et al. 2018). SPRF contains a large amount of proteins and growth factors, inducing the proliferation and migration of human bone marrow derived mesenchymal stem cells (MSCs), osteoblasts and osteoarthritic chondrocytes in vitro in cell culture SPRF has been designed to avoid a number of disadvantageous effects of platelet releasates, since it works through natural coagulation in a single-step preparation process, avoiding issues with the overconcentrated plasma derivatives. Our research goal was to find cellular-level mode of action of SPRF that is already being investigated for degenerative bone pathologies such as OA and osteonecrosis. Specifically, bone marrow lesions are observed in these pathologies due to the loss of regenerative capacity of the cells in this location. We set out to perform preclinical laboratory investigations on monolayer MSC cultures and in their natural niche, in a 3D subchondral bone marrow culture model (BMEs). (Kuten, Simon et al. 2018, Simon, Major et al. 2018, Vacz, Major et al. 2018, Kardos, Simon et al. 2019).

SPRF proved to be surprisingly advantageous and preferred over HSA.

However the skilled person will understand that other protein preparations may be linked to the surface of the DVS-cross-linked HA hydrogels, like those taught above.

Methods to Characterize Hydrogels

Suitable methods to characterize microstructure of the hydrogels are known in the art.

For the texture determination of hydrogels, e.g. freeze-dried hydrogel samples, scanning electron microscopy (SEM) studies can be carried out. Typical magnifications may be e.g. 10 to 2000 times or 20 to 1000 times. Samples shall be coated in advance of the measurement e.g. by a thin inert metal film like gold. In the present examples the structure of the cross-linked gels was examined by SEM. The surface and the cross section of 2% and 5% BDDE and 2% and 5% DVS gels were compared to each other.

Confocal laser scanning microscopy (CLSM) can also carried out to characterize the morphology of hydrogels. Fourier Transform-Infrared (FT-IR) Spectrometer is useful to obtain spectra of the hydrogel samples e.g. between 500 and 4000 cm-1. Mechanical characterization of the gels can be carried out by well-known material science method e.g. as described herein or elsewhere. (Strom, Anna 2015)

The methods are applicable also to characterize the freeze-dried hydrogel samples.

Swelling ratio is the quotient of the swollen and the freeze-dried gels' weight. It is proportional with the degree of cross-linking; a strongly cross-linked hydrogel has a lower water uptake capacity and swells less than a weaker cross-linked gel.

In vitro enzymatic degradation can be examined e.g. with the help of Ehrlich's reagent, which determines the concentration of NAG (N-acetyl-glucosamine), the product of HA degradation. HA gels can be digested with hyaluronidase enzyme e.g. from bovine testis.

Cytotoxicity measurement was performed in a similar way as described in ISO 10993 to ascertain that the cross-linked hydrogels do not contain materials that are harmful to the living cells or hinder proliferation. Similarly, biocompatibility measurements can be made and investigated if cells attach onto the cross-linked gels, as cell adhesion on the surface or inside the structure is an important property of all scaffolds. MSCs cultured on the hydrogels were visualized by live-dead staining.

Uses

The preparation can be used among others for soft tissue implantation, in wound healing applications, internal bleeding or muscle and tendon regenerative material as intended uses.

Hydrogels are widely used in regenerative medicine, e.g as described by Slaughter et al, 2009, Zhang, F. et al., 2011, Schante, C. E et al, 2011, Shimizu, N et al., 2014, Salwowska, N. M et al, 2016, Sahana, T. G et al, 2018, Okabe, K., Y. et al, 2009. etc.

Crosslinked hyaluronic acid hydrogels can be used as scaffolds for soft tissue engineering (J. G. Hardy et al. S. R Van Tomme et al., I. R. Erickson) in cases of soft-tissue defects like congenital malformation, extirpation or trauma (K. Okabe et al.).

The scaffolds can serve as a synthetic extracellular matrix with their high water content and soft structure (B. V. Slaughter et al.) organizing cells into a three-dimensional architecture (J. L. Drury et al.). As these scaffolds closely mimic natural tissues, cells adhere into the three-dimensional network, especially when there are incorporated peptide domains in the hydrogel.

HA hydrogels of the invention can also be used to facilitate wound healing. Normally, the process consists of hemostasis, inflammation, proliferation and remodeling (T. G. Sahana, C. J Deutsch et al.), but in some cases natural wound healing process is hindered or cannot take place and the wound becomes chronic, like diabetic ulcers and pressure ulcers (N. Shimizu et al.), which cannot be recovered without external help. In other cases, like severe burns, large skin damage occurs and therefore an appropriate wound dressing is needed. An ideal wound dressing prevents contamination of the wound and maintains adequate moisture but removes excessive exudates. Wound healing dressing can also be used as drug delivery systems (Boeting et al.)

Hyaluronic acid hydrogels may be excellent wound dressings as they create an advantageous environment for wound healing because of their rheological, hygroscopic and viscoelastic properties. In animal models HA helped re-epithelialization and led to the formation of new soft tissue in case of full-thickness surgical wounds. Although, low molecular weight HA was not reported to have these protective effects (C. L. Wu et al.), it was found to induce angiogenesis following its degradation.

Crosslinked high molecular weight hyaluronic acid gels alone were found to be bioinert (S. Ibhrahim et al.) and cell attachment into these gels is low (A. Ramamurthi et al. ad F. Zhang et al.). However, cell adherence can be promoted by fabricating hybrid HA scaffolds with gelatin, chitosan (D. G. Miranda and Y. Wang et al. Wu, Song et al) or collagen among others which form a hybrid hydrogel or a composite hydrogel. Peptide incorporation into the hydrogel is another way to enhance cell attachment, migration, proliferation, growth and organization. Besides, HA hydrogels can be coated with collagen, extracellular matrix gel, laminin and fibronectin to enhance cellular adhesion.

Blood derived protein polymerization or crosslinking into the gels can be another option to advance cell attachment. Serum from platelet rich fibrin (SPRF, also referred to as hyperacute serum) is a human blood derivative, which is isolated from whole blood without anticoagulants and may be crosslinked covalently to the HA matrix.

Products According to the Invention

Cross-linked HA has been used for longer than 15 years and is considered to be generally well tolerated. HA has hydrophilic nature and is biocompatible.

However, the present invention opens up new or improved application by increasing cross-linked HA materials capability for cell adhesion and recruitment.

The final product according to the invention may be in the form of a film or a scaffold or a powder.

Preferably the product is lyophilized (or freeze-dried). This increases storability. By adding water or electrolyte or buffered solution the product can be reconstituted and applied in the patient.

As a scaffold or graft it may have a sponge-like feature in that it comprises holes or cavities. Thus, the inner structure is similar to that of bone or an actual sponge.

The scaffold or graft products of the invention have a tunable elasticity and rigidity by the ratio of the cross-linker applied. Thereby also the time of degradation on the site of application in the subject's or patient's body can be adjusted.

### EXAMPLES

### 1. Materials and Methods

### 1.1. Hydrogel Preparation Methods

### Method 1—Preparation of Crosslinked HA Hydrogels

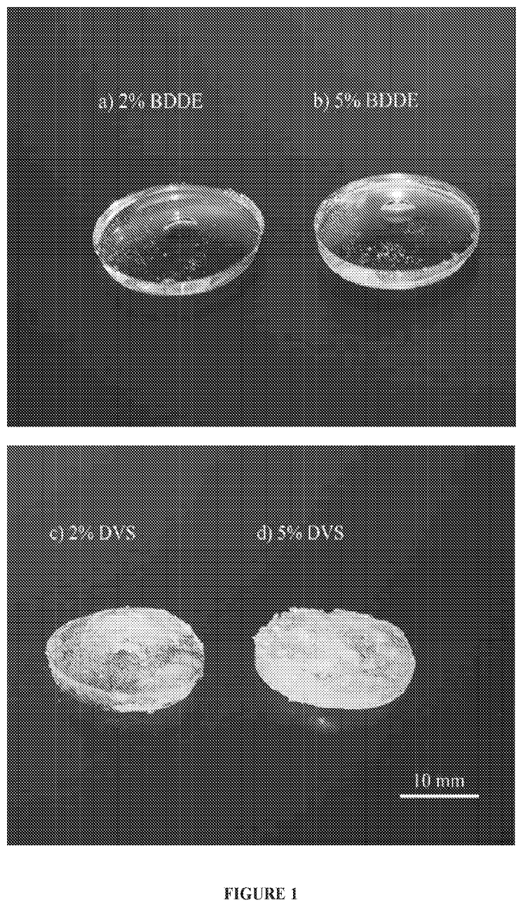

Crosslinked HA hydrogels were prepared using 1.34 MDa freeze-dried sodium hyaluronate from bacterial source (Contipro, Dolní Dobrouč, Czech Republic), butanediol-diglycidyl ether (Sigma-Aldrich, St. Louis, Mo., USA), or divinyl sulfone (abcr, Karlsruhe, Germany) and NaOH to provide alkaline condition required for the crosslinking reaction. The crosslinkers (BDDE or DVS) were used in 2 V/V %, 5 V/V % and 10 V/V %. BDDE or DVS was mixed with 1 ml 1% NaOH (Molar Chemicals) and then added to 133 mg sodium hyaluronate and immediately vortexed until a homogenous gel was formed. The hydrogels were centrifuged at 1700 g for 3 minutes to get flat gels and allowed to crosslink for 48 hours at room temperature in a plastic vial. The crosslinked gels (FIG. 1,) were washed and swollen until equilibration with 80 ml distilled water in three steps, 12 hours each step. (In earlier procedures smaller amount, i.e. 15 ml was applied.) The 10% crosslinker containing gels were more rigid and they moldered during the washing procedure, thus, only the 2 and 5% crosslinker containing gels were further investigated. The washed gels were autoclaved for 20 minutes at 121° C. to get a sterile gel. Sterilized gels were freeze-dried at −55° C. and 5 Pa.

### Method 2—Further Modifying the Freeze-Dried HA Gels

In this method in a second cross-linking step the gel obtained from method 1 has been further modified. Specifically, the sterile, freeze-dried HA gels were further modified by crosslinking SPRF into their structure using DVS, or fibrinogen polymerization to improve cell adhesion on the gels. When preparing SPRF containing gels, each freeze-dried quarter of gel was soaked into 1 ml 5% sterile DVS containing SPRF at pH=12 and crosslinking took place for 24 hours at room temperature. The gels were washed again three times with sterile distilled water for 12 hours each step to remove excess non-reacted DVS. In case of the preparation of fibrin containing gels, 20 μl 1 M CaCl₂ and 20 μl (500 U/ml) thrombin were added to 1 ml cryoprecipitate and it was poured onto the freeze-dried crosslinked HA gels (1 ml recalcined cryoprecipitate was added to each quarter of freeze-dried gel.) The recalcined cryoprecipitate gets absorbed by the gels and the fibrinogen converts into fibrin polymers inside the structure of the HA gels in one hour at room temperature. The whole protein crosslinking and washing procedure occurred under aseptic conditions using sterile filtered reagents, thus there was no need to further sterilize the prepared SPRF and fibrin containing gels.

SPRF and Cryoprecipitate Isolation from Whole Blood

Phlebotomy was used from healthy donors, men and women, aged 24-45 years. 50 ml venous blood was drawn from each donor using a butterfly needle and a syringe. In case of SPRF production, whole blood was poured into a 50 ml centrifuge tube containing 10 g sterile glass beads under a laminar flow hood and centrifuged immediately at 1710 g for 8 minutes to separate red blood cells from serum fraction. Blood clotting was promoted by the glass and the fibrin clot was formed. The tube was centrifuged again at 1710 g until fibrin clot became about 1 cm flat and supernatant was collected, which is SPRF. In case of cryoprecipitate, whole blood was poured into a sterile 50 ml centrifuge tube, which contained 0.215 g sodium citrate dihydrate (Sigma-Aldrich, St. Louis, Mo., USA) dissolved in 0.5 ml saline solution. It was centrifuged at 700 g for 8 minutes and then at 1710 g until the plasma fraction was separated from the red blood cell containing fraction. The plasma was collected and kept at −80° C. for 24 hours and then thawed at 3° C. and centrifuged at 3260 g for 12 minutes. The cryoprecipitate was dissolved in 10 ml plasma, the rest of the supernatant fraction was removed.

Gel Homogenization

4 g of protein crosslinked HA was extended with 1 ml saline solution, the components were homogenized for 5 minutes using a Tissueruptor homogenizer. The homogenized gel was either frozen, freeze-dried or filled in a syringe and frozen.

### 1.2. Swelling Ratio Measurement

Cross-linked, washed and swollen gels were weighed using an analytical balance. The gels were freeze-dried and weighed again. Swelling ratio was calculated with the following formula:

Swelling ratio=M_{swollen gel}/M_{freeze-dried gel}

### 1.3. Enzymatic Degradation Measurement

Enzymatic degradation was determined using Ehrlich's solution (Sigma-Aldrich, St. Louis, Mo., USA), which is a reagent consisting of acetic acid, p-dimethyl amino-benzaldehyde and hydrochloric acid. Ehrlich's solution detects N-acetyl-glucosamine, a product of HA degradation. It was observed, that heated solution of N-acetyl-glucosamine reacting with p-dimethyl amino-benzaldehyde under acidic conditions presents purple color, proportional with the N-acetyl-glucosamine concentration. The intensity of the color can be improved by adding borate to the reaction mix (Morgan and Elson 1934, Reissig, Storminger et al. 1955, Asteriou, Deschrevel et al. 2001).

Quarters of cross-linked 2% BDDE, 5% BDDE, 2% DVS and 5% DVS containing HA hydrogels were soaked into 10 ml 4 mg/mL solution of hyaluronidase from bovine testes, type I-S(Sigma-Aldrich, St. Louis, Mo., USA) and kept at 37° C. on a shaker for 100 hours, while N-acetyl-glucosamine concentration measurement was carried out twice a day with Ehrlich's reagent according to the following protocol: 50 μl of the enzyme solution was mixed with 50 μl borate buffer (4.94 g H₃BO₃, 1.98 g KOH in 100 ml H₂O, pH=9) and placed into boiling water for 3 minutes and allowed to cool down to room temperature for 5 minutes, then 25 μl glacial acetic acid and 25 μl Ehrlich's reagent was added. Absorbance was measured 12 minutes after Ehrlich's reagent was added at 585 nm with a reference wavelength at 750 nm using a PowerWave XS microplate spectrophotometer (BioTek, Winooski, Vt., USA).

### 1.4. Observation of the Structure of the Cross-Linked Hydrogels by Scanning Electron Microscopy

The structure of the gels was examined by a scanning electron microscope (SEM, JEOL JSM-6380LA). Cross-linked gels were prepared as described above and fixed with 2,5% glutaraldehyde for 20 minutes. Dehydration of fixed gels was achieved with increasing concentrations of ethanol (50, 70, 80, 90, 100%, for 5 minutes each step) and treating with hexamethyl disilazane for 5 minutes and dried overnight. The fixed gels were coated with gold (JEOL JFC-1200 Fine Coater, 12 mA, for 20 seconds) and their surface and cross-section were examined with SEM.

### 1.5. SPRF Isolation from Whole Blood

Phlebotomy occurred under IRB approval (IRB approval number 33106-1/2016/EKU, 12.07.2016.) from healthy donors. 50 ml venous blood was drawn from each donor using a butterfly needle and a syringe. Whole blood was poured into a 50 ml centrifuge tube containing 10 g sterile glass beads under a Class II laminar flow hood and centrifuged immediately at 1710 g for 8 minutes to separate red blood cells from serum fraction. Blood clotting was promoted by the glass and the fibrin clot was formed. The tube was centrifuged again at 1710 g until fibrin clot became about 1 cm flat and supernatant was collected, which is SPRF.

### 1.6. Protein Cross-Linking into the Hydrogels

The autoclaved, freeze-dried gels were further modified by cross-linking HSA (CSL Behring, King of Prussia, Pa., USA) or SPRF into their structure using DVS to improve cell adhesion on the gels. Each freeze-dried quarter was soaked in 1 ml 5% sterile DVS containing SPRF or HSA solution (the concentration of the HSA solution was normalized to the protein content of SPRF) at pH=12 and cross-linking took place for 24 hours at room temperature. The gels were washed again three times with sterile distilled water for 12 hours in each step to remove excess non-reacted DVS. The whole protein cross-linking and washing procedure occurred under aseptic conditions using sterile filtered reagents, thus there was no need to further sterilize the prepared SPRF and HSA containing gels.

### 1.7. Cytotoxicity and Biocompatibility of the Hydrogels

### 1.7.1. Mesenchymal Stem Cell Culturing

All cell culture procedures were carried out in a sterile laminar flow tissue culture hood. Bone marrow derived mesenchymal stem cells (MSCs, ATCC, Manassas, Va., USA) were cultured in T-75 TC treated culture flasks in an incubator at 37° C., 5% CO₂ and 95% humidity. MSCs were maintained in stem cell medium: Dulbecco's modified Eagle's medium containing 4.5 g/L glucose and L-glutamine (Lonza, Basel, Switzerland) supplemented with 10% fetal bovine serum (EuroClone, Pero, Italy), 1% Penicillin-Streptomycin (Sigma-Aldrich, St. Louis, Mo., USA) and 0.75 ng/mL basic fibroblast growth factor (Sigma-Aldrich, St. Louis, Mo., USA). Culture medium was refreshed three times a week.

### 1.7.2. Cytotoxicity Measurement with XTT

Cytotoxicity measurements were performed to examine if cross-linked HA gels are cytotoxic as they may contain any excess of toxic reagents which can be released to their environment. Mesenchymal stem cells (4p) were seeded onto the bottom of 12 well plates in a density of 5000 cells/well in 2 ml stem cell medium. 3-4 mm³ pieces of the sterile and washed HA gels and also SPRF and HSA containing cross-linked HA gels were washed with 1.5 ml stem cell medium for 4 hours. On the first day the gel pieces were placed into the medium of the MSCs, while there were 3 cell containing wells without gel in them as controls. The medium was refreshed twice a week. The viability of the MSCs was measured on the seventh day with the help of Cell Proliferation Kit II. (XTT; Roche, Mannheim, Germany) according to the manufacturer's instructions. The difference between the gel containing and control wells shows cytotoxicity of the cross-linked gels.

### 1.7.3. Biocompatibility Test by Live/Dead Staining

Biocompatibility tests were accomplished to investigate if MSCs adhere and proliferate on the cross-linked hydrogels. Sterile and washed 3-4 mm³ pieces of the cross-linked gels were washed with 1.5 ml stem cell medium for 4 hours. On the first day 25 000 MSCs (4p) were seeded onto the gels on 24 well low attachment plates in stem cell medium. The medium was refreshed twice a week. On the 14^{th} day the attaching cells were visualized on the gels by live-dead staining. The gels were washed three times with PBS and stained in PBS containing 1 μM Calcein-AM (Invitrogen, Carlsbad, Calif., USA), 4 μg/mL ethidium homodimer (Invitrogen, Carlsbad, Calif., USA) and 20 μg/mL Hoechst (Invitrogen, Carlsbad, Calif., USA) for 30 minutes. The gels were washed again three times with PBS and images were taken by an inverse fluorescent Nikon Eclipse Ti2 microscope.

### 1.8. Statistical Analysis

One-way analysis of variance (ANOVA) with Tukey post hoc test and Kruskal-Wallis test with Dunn's post hoc test were performed with D'Agostino & Pearson omnibus normality test to compare the means of groups using Prism 7 software. The significance level was p<0.05 and data are presented as mean±SEM.

### Example 2

### 2.1. Swelling Ratio

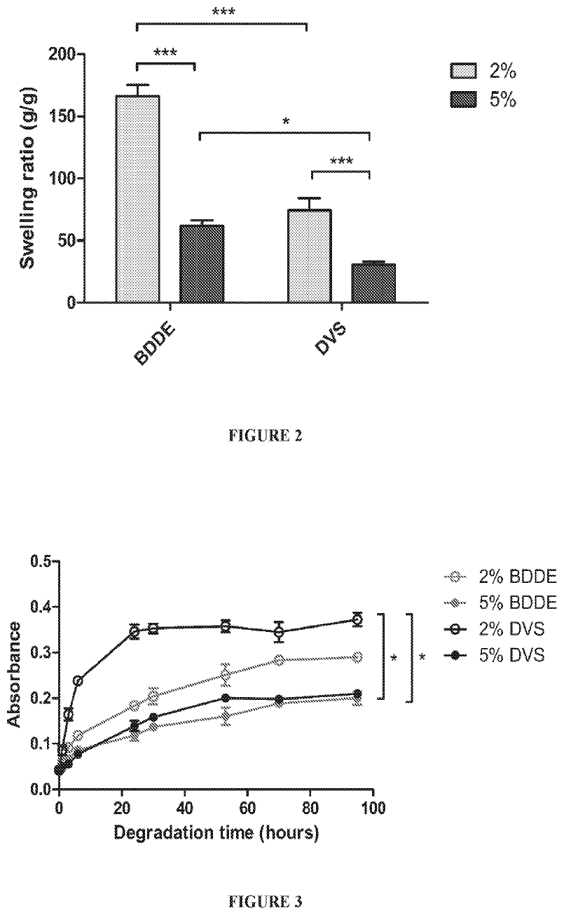

Swelling ratio is the quotient of the swollen and the freeze-dried gels' weight. It is proportional with the degree of cross-linking; a strongly cross-linked hydrogel has a lower water uptake capacity and swells less than a weaker cross-linked gel. One-way analysis of variance (ANOVA) with Tukey post hoc test was performed and it was observed that the gels containing 2% cross-linker had significantly higher swelling ratio than 5% DVS or BDDE containing gels. In addition, the gels cross-linked with DVS were significantly less swollen than BDDE gels containing the same amount of cross-linker, consequently, their cross-linking density is higher (FIG. 2).

### 2.2. Enzymatic Degradation

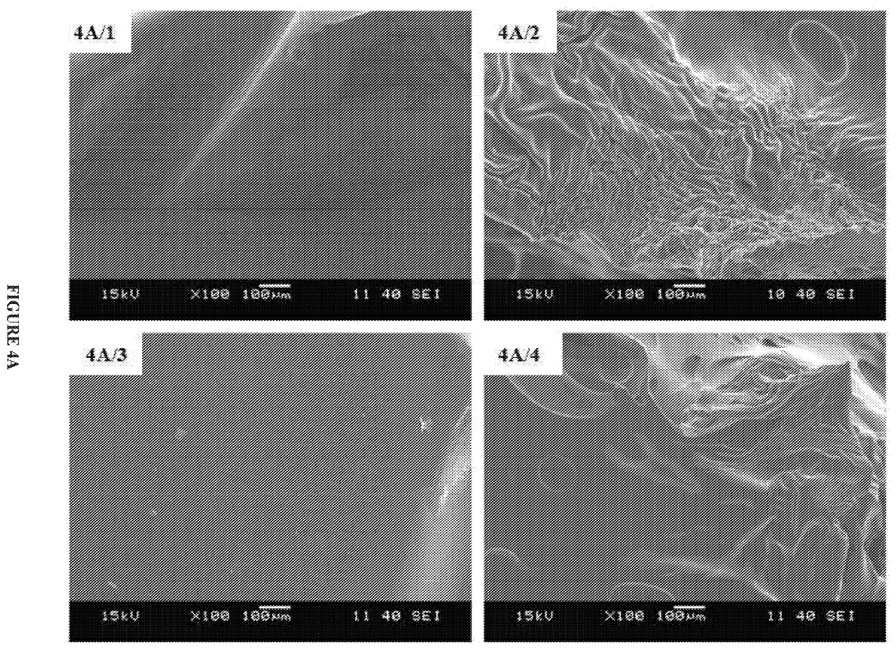

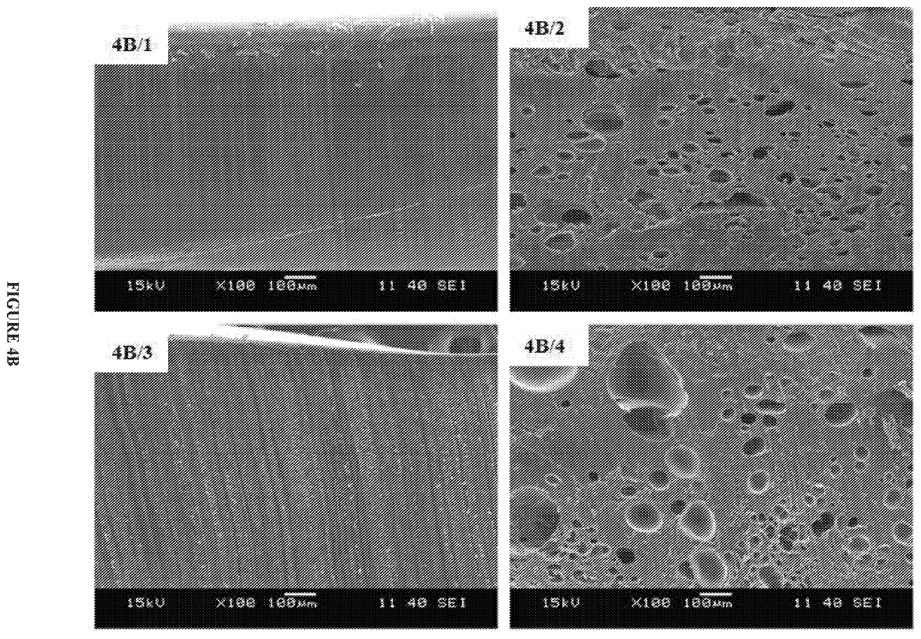

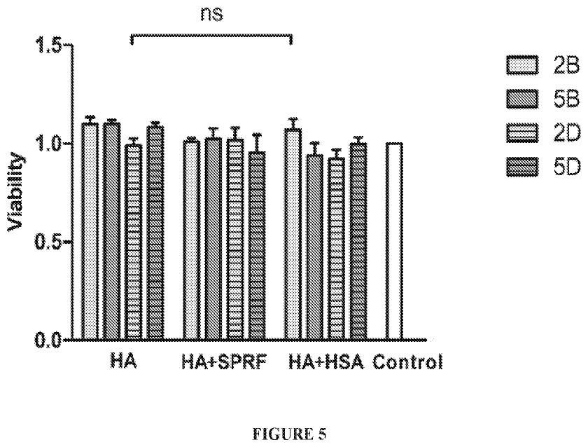

In vitro enzymatic degradation was examined with the help of Ehrlich's reagent, which determines the concentration of NAG (N-acetyl-glucosamine), the product of HA degradation (FIG. 3). HA gels were digested with hyaluronidase enzyme from bovine testis. NAG concentration, which is proportional to HA degradation increased with time, but after 70 hours the degradation slowed down, probably because enzyme activity decreased. The 2% DVS containing gel was found to be degrading the fastest, significant difference was observed between 2% DVS and 5% DVS and between 2% DVS and 5% BDDE containing gels using One-way analysis of variance (ANOVA) with Tukey post hoc test, but after 30 hours the N-acetyl-glucosamine concentration reached a final value. 5% BDDE and 5% DVS gels were the most resistant to enzymatic degradation, as their cross-linking density was higher, which also affects enzymatic degradation. None of the gel quarters were fully digested after 100 hours, the water insoluble gel pieces were still visible (FIG. 3).

### 2.3. Structure

The structure of the cross-linked gels was examined by SEM. The surface and the cross section of 2% and 5% BDDE and 2% and 5% DVS gels were compared to each other. The surface of both BDDE gels were found to be extremely smooth, while DVS gels were more furrowed and rougher. The cross section of BDDE gels was also smooth and dense, while DVS gels contained small bubbles in their structure probably because of technical reasons. The bubbles can originate from the preparation of the gels as DVS reacts very fast and it started to cross-link the hyaluronic acid during the vortexing step and the bubbles stuck in the structure. BDDE reacts slower and in this case the bubbles could be removed during centrifugation (FIG. 4).

### 2.4. Cytotoxicity

Cytotoxicity measurement was performed to ascertain that the cross-linked hydrogels do not contain materials that are harmful to the living cells or hinder proliferation. Cross-linker reagents, as BDDE and DVS are toxic and if the gels contain unreacted amounts of them, then they can cause cell death. Thus, the hydrogels were washed several times after cross-linking. Cytotoxicity test showed that the viability of cells cultured on the bottom of the well in the presence of differently cross-linked hydrogels was as high as the viability of control cells, which were cultured in the wells without hyaluronic acid gels. No significant difference was detected performing Kruskal-Wallis with Dunn's post hoc test; therefore, it was observed, that none of the cross-linked gels was cytotoxic (FIG. 5).

### 2.5. Biocompatibility

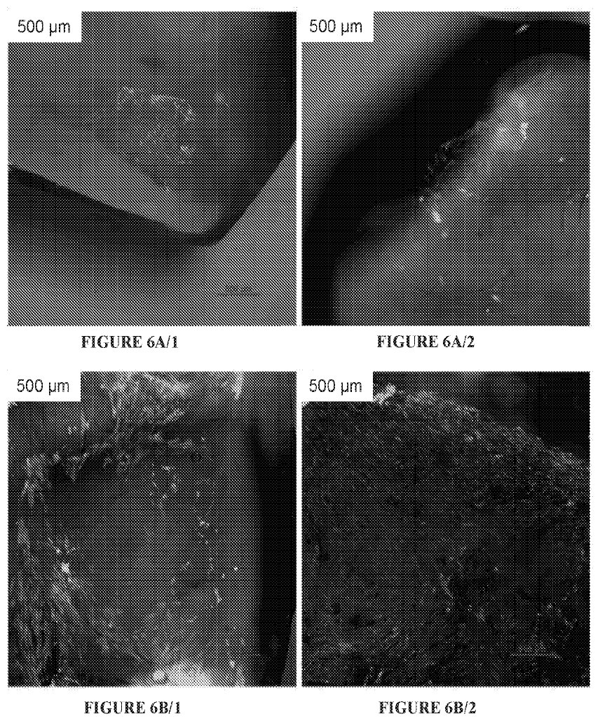

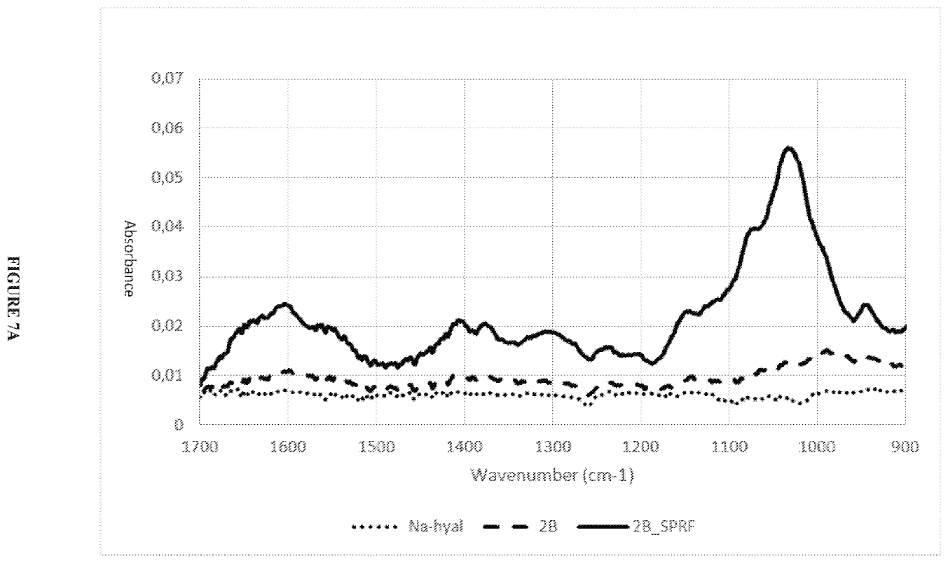

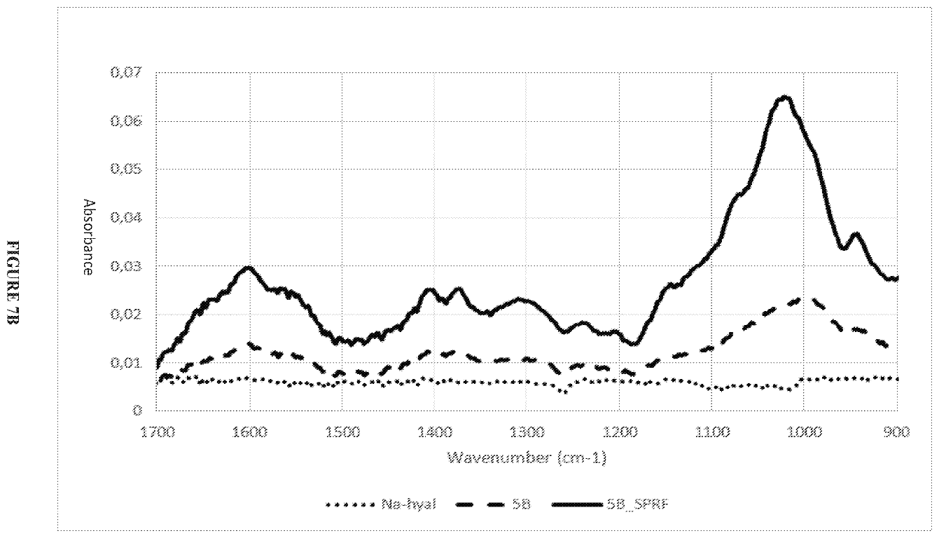

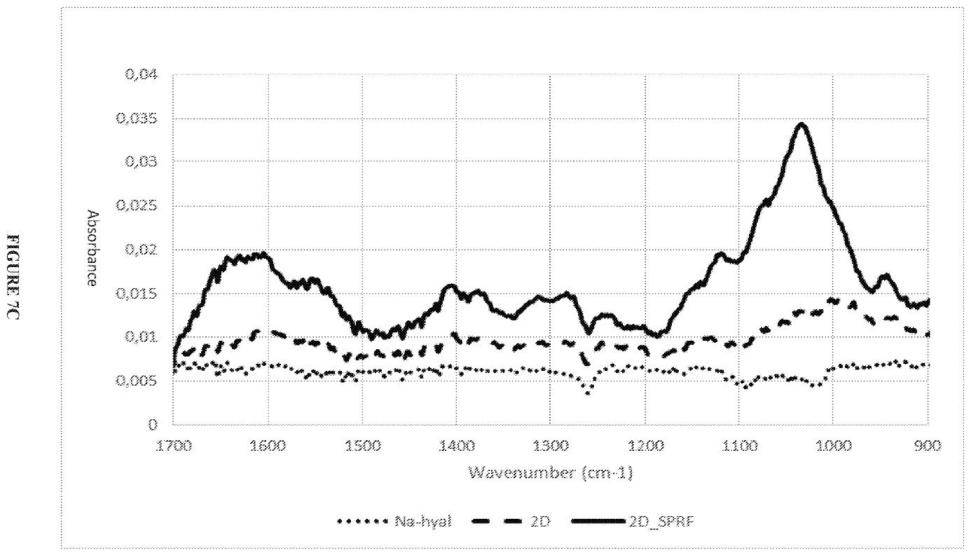

It was investigated if cells attach onto the cross-linked gels, as cell adhesion on the surface or inside the structure is an important property of all scaffolds. MSCs cultured on the hydrogels were visualized by live-dead staining. On the HA gels, which did not contain cross-linked proteins no cells could be observed, which is in good accordance with previous studies (Ramamurthi and Vesely 2002, Ibrahim, Kang et al. 2010, Zhang, He et al. 2011). However, on the SPRF or HSA containing hydrogels attached living cells could be visualized. There were no dead cells on any of the gels, probably because dead cells can easily be washed down during the staining procedure. The nuclei were visible after staining, but Hoechst could not be removed from the gels and the blue background was strong even after careful washing, thus this channel is not shown in the pictures. It was observed, that MSCs attached and proliferated only on 2% DVS and 5% DVS hydrogels, both if HSA or SPRF was cross-linked inside the gels, although, on SPRF gels more cells could be seen and cell attachment was stronger. No cells could be seen on BDDE cross-linked hydrogels, probably because of the smooth surface of these gels. Besides, more MSCs could be detected on the cut edges of DVS gels, which also suggests, that cell attachment depends on the surface structure, and it is stronger on rough surfaces (FIG. 6).

### Example 3

### Treatment of Mice with Hydrogel

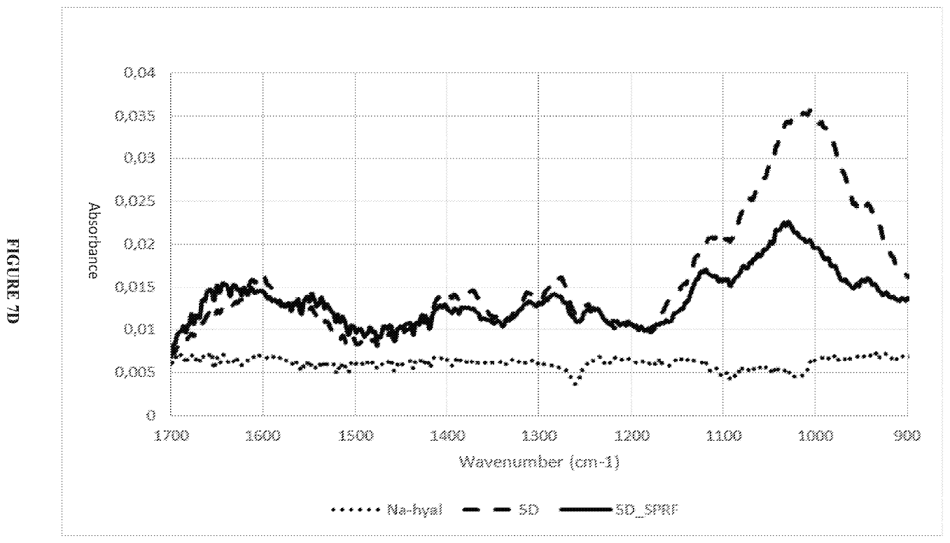

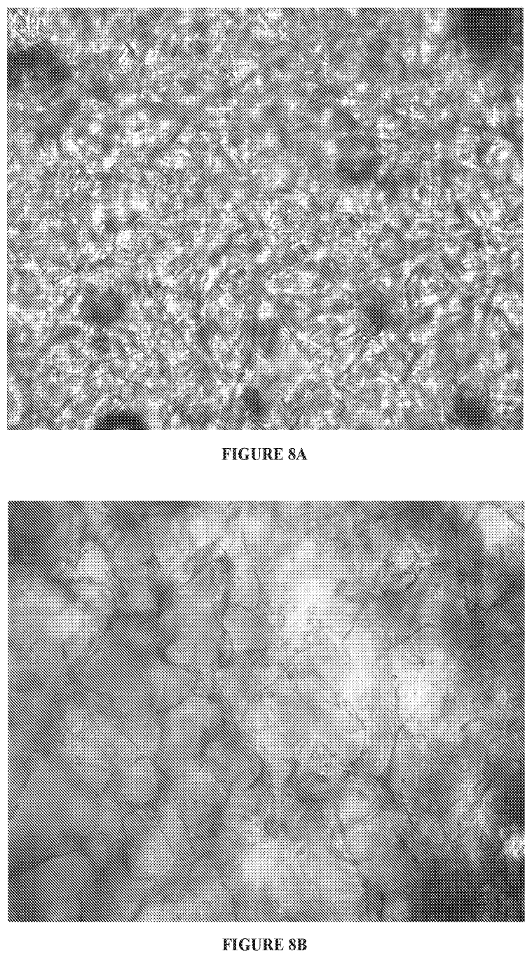

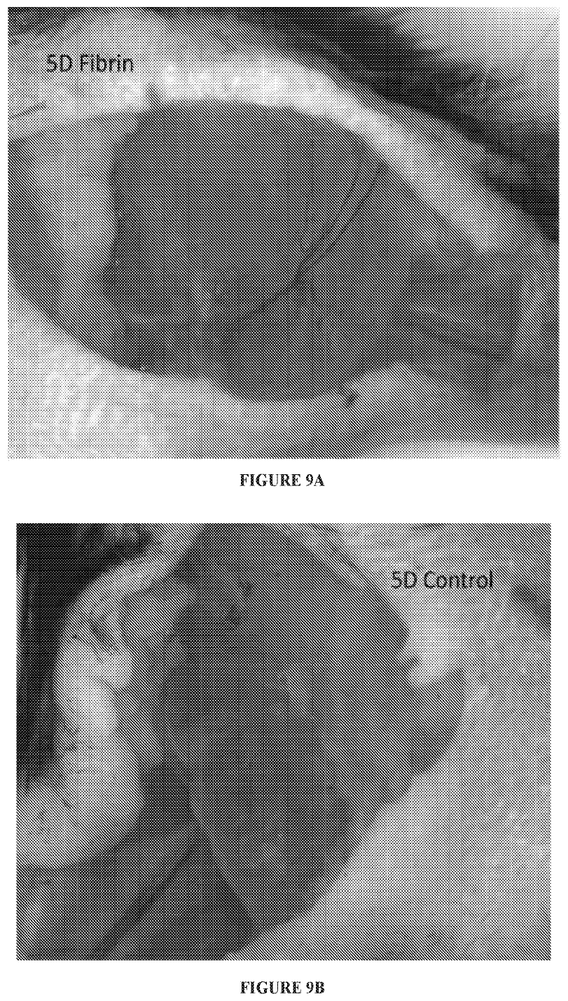

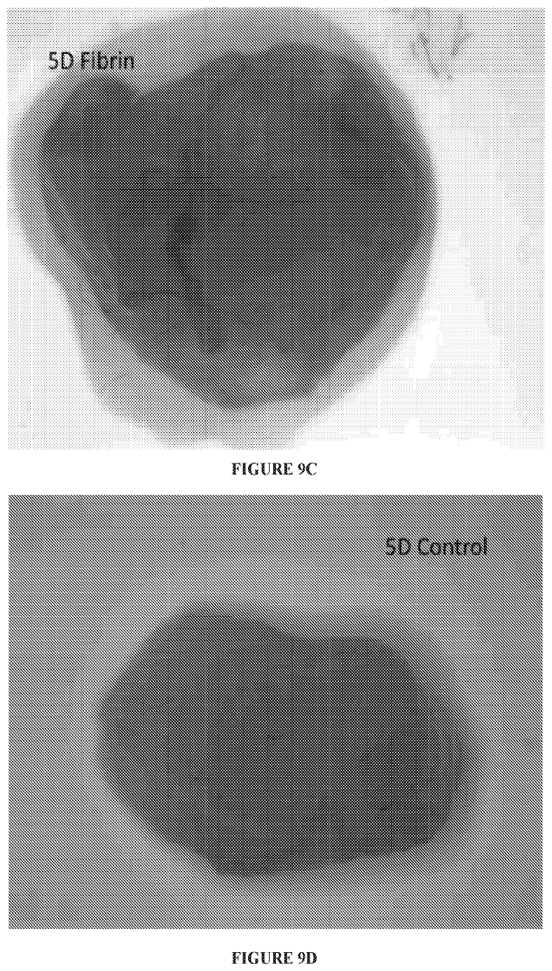

Homogenized gel suspension prepared according to Method 3, as described in Example 1, section 1.1. (FIG. 7,A) has been injected into mice as follows: 200 ul of homogenized gel was injected subcutaneously in both the left and right inguinal region of black six type 6 week old mice. The right injection site contained homogenized HA, which was crosslinked with fibrin, the left site contained only homogenized HA, (type SD, that stands for HA prepared using 5% DVS as crosslinker). The mice were sacrificed 12 weeks later, the consistency, vascularization and weight was investigated, it was found that generally the initial gel still contained crystalline type HA parts (FIG. 7.A), but the implants 12 weeks later were filled with connective tissue and vascularization already took place (FIG. 7.B). Surprisingly, the fibrin containing implants contained a larger vascularized ratio (FIGS. 8A and C, type SD “Fibrin”), compared to the gels, which did not contain fibrin (FIGS. 8B and D, type SD “control”).

### INDUSTRIAL APPLICABILITY

The protein-cross-linked HA hydrogel of the invention is particularly useful in regenerative medicine, e.g. for soft tissue implantation, wound healing, internal bleeding or muscle and tendon regenerative material, etc.

### REFERENCES

Asteriou, T., B. Deschrevel, B. Delpech, P. Bertrand, F. Bultelle, C. Merai and J. C. Vincent (2001). “An improved assay for the N-acetyl-D-glucosamine reducing ends of polysaccharides in the presence of proteins.” Anal Biochem 293(1): 53-59.
Boeting, J. S., Matthews, K. H., Stevens, H. N. E., Eccleston, G. M. J. Wound healing dressings and drug delivery systems: A review Pharm Sci (2008) 97(8) 2892-2923
Choukroun, J. Reduction of relative centrifugation force within injectable platelet-rich-fibrin (PRF) concentrates advances patients' own inflammatory cells, platelets and growth factors: the first introduction to the low speed centrifugation concept. Eur J Trauma Emerg Surg (2018) 44:87-95
De Boulle, Koenraad, Richard Glogau, Taro Kono, Myooran Nathan, Ahmet Tezel, Jean-Xavier Roca-Martinez, Sumit Paliwal And Dimitrios Stroumpoulis (2013). “A Review of the Metabolism of 1,4-Butanediol Diglycidyl Ether-Cross-linked Hyaluronic Acid Dermal Fillers.” Dermatol Surg 39:1758-1766.
Deutsch, C. J., D. M. Edwards and S. Myers (2017). “Wound dressings.” Br J Hosp Med (Lond) 78(7): C103-c109.
Drury, J. L. and D. J. Mooney (2003). “Hydrogels for tissue engineering: scaffold design variables and applications.” Biomaterials 24(24): 4337-4351.
Fallacara, A., E. Baldini, S. Manfredini and S. Vertuani (2018). “Hyaluronic Acid in the Third Millennium.” Polymers 10(7): 701.
Franchini M. Plasma-derived versus recombinant Factor VIII concentrates for the treatment of haemophilia A: recombinant is better. Blood Transfus. 2010 October; 8(4):292-6
Ghosh, K., X. Z. Shu, R. Mou, J. Lombardi, G. D. Prestwich, M. H. Rafailovich and R. A. Clark (2005). “Rheological characterization of in situ cross-linkable hyaluronan hydrogels.” Biomacromolecules 6(5): 2857-2865.
Gokila, S., T. Gomathi, K. Vijayalakshmi, F. A. AAlshahrani, S. Anil and P. N. Sudha (2018). “Development of 3D scaffolds using nanochitosan/silk-fibroin/hyaluronic acid biomaterials for tissue engineering applications.” Int J Biol Macromol 120(Pt A): 876-885.
Hardy, J. G., P. Lin and C. E. Schmidt (2015). “Biodegradable hydrogels composed of oxime cross-linked poly(ethylene glycol), hyaluronic acid and collagen: a tunable platform for soft tissue engineering.” J Biomater Sci Polym Ed 26(3): 143-161.
Hennink, W. E. and C. F. van Nostrum (2002). “Novel cross-linking methods to design hydrogels.” Adv Drug Deliv Rev 54(1): 13-36.
Ibrahim, S., Q. K. Kang and A. Ramamurthi (2010). “The impact of hyaluronic acid oligomer content on physical, mechanical, and biologic properties of divinyl sulfone-cross-linked hyaluronic acid hydrogels.” J Biomed Mater Res A 94(2): 355-370.
Jooybar, E., M. J. Abdekhodaie, M. Alvi, A. Mousavi, M. Karperien and P. J. Dijkstra (2019). “An injectable platelet lysate-hyaluronic acid hydrogel supports cellular activities and induces chondrogenesis of encapsulated mesenchymal stem cells.” Acta Biomater 83: 233-244.
Kanaide H. Cross-linking of fibrinogen and fibrin by fibrin-stablizing factor (factor XIIIa). J Lab Clin Med. 1975 April; 85(4):574-97.
Kardos, D., I. Hornyak, M. Simon, A. Hinsenkamp, B. Marschall, R. Vardai, A. Kallay-Menyhard, B. Pinke, L. Meszaros, O. Kuten, S. Nehrer and Z. Lacza (2018). “Biological and Mechanical Properties of Platelet-Rich Fibrin Membranes after Thermal Manipulation and Preparation in a Single-Syringe Closed System.” Int J Mol Sci 19(11).
Kardos, D., M. Simon, G. Vacz, A. Hinsenkamp, T. Holczer, D. Cseh, A. Sarkozi, K. Szenthe, F. Banati, S. Szathmary, S. Nehrer, O. Kuten, M. Masteling, Z. Lacza and I. Hornyak (2019). “The Composition of Hyperacute Serum and Platelet-Rich Plasma Is Markedly Different despite the Similar Production Method.” Int J Mol Sci 20(3).
Kenne, L., S. Gohil, E. M. Nilsson, A. Karlsson, D. Ericsson, A. Helander Kenne and L. I. Nord (2013). “Modification and cross-linking parameters in hyaluronic acid hydrogels—definitions and analytical methods.” Carbohydr Polym 91(1): 410-418.
Kuten, O., M. Simon, I. Hornyak, A. De Luna-Preitschopf, S. Nehrer and Z. Lacza (2018). “The Effects of Hyperacute Serum on Adipogenesis and Cell Proliferation of Mesenchymal Stromal Cells.” Tissue Eng Part A 24(11-12): 1011-1021.
La Gatta, A., C. Schiraldi, A. Papa and M. De Rosa (2011). “Comparative analysis of commercial dermal fillers based on cross-linked hyaluronan: Physical characterization and in vitro enzymatic degradation.” Polymer Degradation and Stability 96(4): 630-636.
Lai, J. Y. (2014). “Relationship between structure and cytocompatibility of divinyl sulfone cross-linked hyaluronic acid.” Carbohydr Polym 101: 203-212.
Miranda, D. G., S. M. Malmonge, D. M. Campos, N. G. Attik, B. Grosgogeat and K. Gritsch (2016). “A chitosan-hyaluronic acid hydrogel scaffold for periodontal tissue engineering.” J Biomed Mater Res B Appl Biomater 104(8): 1691-1702.
Morgan, W. T. and L. A. Elson (1934). “A colorimetric method for the determination of N-acetylglucosamine and N-acetylchrondrosamine.” Biochem J 28(3): 988-995.
Neuman, M. G., R. M. Nanau, L. Oruna-Sanchez and G. Coto (2015). “Hyaluronic acid and wound healing.” J Pharm Pharm Sci 18(1): 53-60.
Okabe, K., Y. Yamada, K. Ito, T. Kohgo, R. Yoshimi and M. Ueda (2009). “Injectable soft-tissue augmentation by tissue engineering and regenerative medicine with human mesenchymal stromal cells, platelet-rich plasma and hyaluronic acid scaffolds.” Cytotherapy 11(3): 307-316.
Powell J S. Recombinant factor VIII in the management of hemophilia A: current use and future promise. Ther Clin Risk Manag. 2009 April; 5(2):391-402
Ramamurthi, A. and I. Vesely (2002). “Smooth muscle cell adhesion on cross-linked hyaluronan gels.” J Biomed Mater Res 60(1): 195-205.
Reissig, J. L., J. L. Storminger and L. F. Leloir (1955). “A modified colorimetric method for the estimation of N-acetylamino sugars.” J Biol Chem 217(2): 959-966.
Sahana, T. G. and P. D. Rekha (2018). “Biopolymers: Applications in wound healing and skin tissue engineering.” Mol Biol Rep 45(6): 2857-2867.
Sall, I. and G. Férard (2007). “Comparison of the sensitivity of 11 cross-linked hyaluronic acid gels to bovine testis hyaluronidase.” Polymer Degradation and Stability 92(5): 915-919.
Salwowska, N. M., K. A. Bebenek, D. A. Zadlo and D. L. Wcislo-Dziadecka (2016). “Physiochemical properties and application of hyaluronic acid: a systematic review.” J Cosmet Dermatol 15(4): 520-526.
Satin, Alexander, M, Norelli, Jolanta B., Sgaglione, Nicholas A. and Grandel, Daniel A. Grande Cartilage 1-10, 2019
Schanté, C. E., G. Zuber, C. Berlin and T. F. Vandamme (2011). “Chemical modifications of hyaluronic acid for the synthesis of derivatives for a broad range of biomedical applications.” Carbohydrate Polymers 85(3): 469-489.
Sheila Maiz-Fernández et al. Synthesis and Characterization of Covalently Cross-linked pH-Responsive Hyaluronic Acid Nanogels: Effect of Synthesis Parameters. Polymers 2019, 11, 742
Shimizu, N., D. Ishida, A. Yamamoto, M. Kuroyanagi and Y. Kuroyanagi (2014). “Development of a functional wound dressing composed of hyaluronic acid spongy sheet containing bioactive components: evaluation of wound healing potential in animal tests.” J Biomater Sci Polym Ed 25(12): 1278-1291.

Shimojo, AAM. (2015) The Performance of Cross-linking with Divinyl Sulfone as Controlled by the Interplay Between the Chemical Modification and Conformation of Hyaluronic Acid. J. Braz. Chem. Soc., 26(3), 506-512.

Simon, M., B. Major, G. Vacz, O. Kuten, I. Hornyak, A. Hinsenkamp, D. Kardos, M. Bago, D. Cseh, A. Sarkozi, D. Horvathy, S. Nehrer and Z. Lacza (2018). “The Effects of Hyperacute Serum on the Elements of the Human Subchondral Bone Marrow Niche.” Stem Cells Int 2018: 4854619.
Skaliczki, Gábor (2013) Serum albumin enhances bone healing in a nonunion femoral defect model in rats: a computer tomography micromorphometry study Orthop. April; 37(4): 741-745.
Slaughter, B. V., S. S. Khurshid, O. Z. Fisher, A. Khademhosseini and N. A. Peppas (2009). “Hydrogels in regenerative medicine.” Adv Mater 21(32-33): 3307-3329.
Ström, A Schuster, E Goh S M. (2014) Rheological characterization of acid pectin samples in the absence and presence of monovalent ions Carbohydrate polymers. 113, 336-343
Suner, S. S., S. Demirci, B. Yetiskin, R. Fakhrullin, E. Naumenko, O. Okay, R. S. Ayyala and N. Sahiner (2019). “Cryogel composites based on hyaluronic acid and halloysite nanotubes as scaffold for tissue engineering.” Int J Biol Macromol 130: 627-635.
Turley, E. A., P. W. Noble and L. Y. Bourguignon (2002). “Signaling properties of hyaluronan receptors.” J Biol Chem 277(7): 4589-4592.
Vacz, G., B. Major, D. Gaal, L. Petrik, D. B. Horvathy, W. Han, T. Holczer, M. Simon, J. M. Muir, I. Hornyak and Z. Lacza (2018). “Hyperacute serum has markedly better regenerative efficacy than platelet-rich plasma in a human bone oxygen-glucose deprivation model.” Regen Med 13(5): 531-543.
Van Tomme, S. R., G. Storm and W. E. Hennink (2008). “In situ gelling hydrogels for pharmaceutical and biomedical applications.” International Journal of Pharmaceutics 355(1): 1-18.
Weisel, J W. Mechanisms of fibrin polymerization and clinical implications. Blood. 2013 Mar. 7; 121(10): 1712-1719.
Wu, C. L., H. C. Chou, J. M. Li, Y. W. Chen, J. H. Chen, Y. H. Chen and H. L. Chan (2013). “Hyaluronic acid-dependent protection against alkali-burned human corneal cells.” Electrophoresis 34(3): 388-396.
Wu, Song and Liang Deng, Hanson Hsia, Kai Xu, Yu He, Qiangru Huang, Yi Peng, Zhihua Zhou, Cheng Peng Evaluation of gelatin-hyaluronic acid composite hydrogels for accelerating wound healing. Journal of Biomaterials Applications (2017) 31(10) 1380-1390.
Zhang, F., C. He, L. Cao, W. Feng, H. Wang, X. Mo and J. Wang (2011). “Fabrication of gelatin-hyaluronic acid hybrid scaffolds with tunable porous structures for soft tissue engineering.” International Journal of Biological Macromolecules 48(3): 474-481.
Zhao, N., X. Wang, L. Qin, Z. Guo and D. Li (2015). “Effect of molecular weight and concentration of hyaluronan on cell proliferation and osteogenic differentiation in vitro.” Biochem Biophys Res Commun 465(3): 569-574.

1. A method for the preparation of a cross-linked hyaluronic acid hydrogel and having blood-derived proteins cross-linked into the structure of said hydrogel, said method comprising
providing a hyaluronic acid (HA) solution,
contacting said HA solution with a first cross-linker to provide a cross-linking reaction mixture, said first cross-linker being a cross-linker acting on hydroxyl groups,
cross-linking the HA by a first cross-linker to form a cross-linked HA hydrogel in a first cross-linking step,
optionally carrying out a first processing step for processing the cross-linked gel hydrogel,
preferably freeze-drying the cross-linked HA hydrogel,
contacting a blood-derived protein composition with the cross-linked HA hydrogel,
cross-linking the blood-derived protein by a second cross-linker into the hydrogel to form a protein-cross-linked hydrogel,
optionally carrying out a second processing step for processing the protein-cross-linked hydrogel.

2. The method according to claim 1 said method comprising freeze-drying the cross-linked HA hydrogel from −100 to −20° C. at 0.2 to 20 Pa.

3. The method according to claim 1 said method comprising one or more of the following steps:
HA is cross-linked by the first cross-linker in a pre-determined three dimensional size, in particular in a film, block or spherical shape,
in the first processing step processing the cross-linked hydrogel comprises washing, equilibrating and/or sterilizing the hydrogel, e.g, sterilization using dry or wet heat, EtO or gamma irradiation,
the cross-linked HA hydrogel is freeze-dried.
cross-linking a blood-derived protein into the hydrogel to form a protein-cross-linked hydrogel,
the second processing step for processing the protein-cross-linked hydrogel, comprises washing and shaping including milling, cutting, homogenization and freeze-drying the hydrogel.

4. The method according to claim 1 wherein
said first cross-linker is selected from the group consisting of 1,4 butanediol diglycidyl ether (BDDE) or divinyl sulfone (DVS), preferably DVS.

5. (canceled)

6. The method according to claim 1 wherein
the blood-derived protein is a plasma-derived preparation and the second cross-linker is a blood-clotting factor or multiple blood-clotting factors inherently present in the preparation.

7. The method according to claim 1 wherein
the HA solution comprises HA having a molecular weight (MW) of 0.1-10 MDa,
the first cross-linking reaction mixture comprises a cross-linker in 1 to 15% (weight percent or W/V percent), wherein preferably the cross-linker is BDDE or DVS, particularly preferably DVS, and alkaline pH is provided in the cross-linking reaction mixture,
the first cross-linking is carried out preferably for 12 to 96 hours,
freeze-drying of the cross-linked hydrogel is carried out from −100 to −20° C., at 0.2 to 20 Pa.

8. The method according to claim 6 wherein
the plasma derived preparation is selected from the group consisting of
a plasma preparation, preferably selected from activated plasma, pooled plasma and antibody-reduced plasma,
a serum preparation, preferably selected from coagulated whole blood, platelet-rich plasma and serum fraction of PRF (SPRF or hyperacute serum),
an isolated plasma protein composition, preferably selected from serum-albumin, serum albumin plus regulatory proteins, serum albumin plus fibrinogen and blood-clotting factors, regulatory proteins plus fibrinogen and blood-clotting factors, serum, plasma, cryoprecipitate; optionally wherein at least a part of the plasma proteins is/are recombinant protein(s).

9. The method according to claim 8 wherein the blood-derived protein composition is a serum fraction of PRF (SPRF or hyperacute serum).

10. The method according to claim 8 wherein the blood-derived protein composition is a cryoprecipitate, or a fibrinogen preparation.

11. A protein-cross-linked hyaluronic acid hydrogel (protein-cross-linked HA hydrogel) having blood-derived proteins cross-linked into the structure of said hydrogel which is obtained by the method according to claim 1.

12. (canceled)

13. The protein-cross-linked HA hydrogel according to claim 11, wherein the cross-linked hydrogels are formed or shaped or moulded or are in the form of a graft, shaped prostheses, membrane, filler, wound cover etc., wherein the gels are washed and preferably the washed gels are sterilized, preferably autoclaved, and preferably freeze-dried.

14. The protein-cross-linked HA hydrogel according to claim 11 wherein
said first cross-linker is selected from the group consisting of 1,4 butanediol diglycidyl ether (BDDE) or divinyl sulfone (DVS), preferably DVS.

15. (canceled)

16. The protein-cross-linked HA hydrogel according to claim 11 wherein
the blood-derived protein is a plasma-derived preparation and the second cross-linker is a blood-clotting factor or multiple blood-clotting factors inherently present in the preparation.

17. The protein-cross-linked HA hydrogel according to claim 16 wherein
the plasma-derived preparation is selected from the group consisting of
a plasma preparation, preferably selected from activated plasma, pooled plasma and antibody-reduced plasma,
a serum preparation, preferably selected from coagulated whole blood, platelet-rich plasma and serum fraction of PRF (SPRF or hyperacute serum),
an isolated plasma protein composition, preferably selected from serum-albumin, serum albumin plus regulatory proteins, serum albumin plus fibrinogen and blood-clotting factors, regulatory proteins plus fibrinogen and blood-clotting factors, serum, plasma, cryoprecipitate; optionally wherein at least a part of the plasma proteins is/are recombinant protein(s).

18. The protein-cross-linked HA hydrogel according to claim 17 wherein the blood-derived protein composition is a serum fraction of PRF (SPRF or hyperacute serum).

19. The protein-cross-linked HA hydrogel according to claim 17 wherein the blood-derived protein composition is a cryoprecipitate, or a fibrinogen preparation.

20. The protein-cross-linked HA hydrogel wherein the hydrogel is obtained by a method according to claim 2 and the blood-derived protein is distributed inside the hydrogel.

21. A method of treatment by using the protein-cross-linked HA hydrogel as obtained by the method of claim 1 in regenerative medicine, wherein said protein-cross-linked HA hydrogel is administered, preferably grafted or implanted into a mammalian, preferably human subject at the site of his/her body to be subjected to regenerative treatment.

22. The method of treatment according to claim 21 wherein the protein-cross-linked HA hydrogel is used for soft tissue implantation, wound healing, internal bleeding or muscle and tendon regenerative material.

23. The method of treatment according to claim 21 wherein said protein-cross-linked HA hydrogel is grafted or implanted in the form of a moulded pre-formed formulation.

24. The method of treatment according to claim 21 wherein said protein-cross-linked HA hydrogel is grafted or implanted by injecting it in the form of a suspension.

25. A method of treatment by using the protein-cross-linked HA hydrogel of claim 11 in regenerative medicine, wherein said protein-cross-linked HA hydrogel is administered, preferably grafted or implanted into a mammalian, preferably human subject at the site of his/her body to be subjected to regenerative treatment.
